(19) United States
(12) Patent Application Publication
(10) Pub. No.: US 2015/0209390 A1
(43) Pub. Date: Jul. 30, 2015

(54) METHOD AND PHARMACEUTICAL COMPOSITION COMPRISING ADIPOSE STEM CELL EXTRACT FOR TREATING OR PREVENTING NEUROLOGIC DISEASE

(71) Applicant: SNU R&DB FOUNDATION, Seoul (KR)

(72) Inventors: Kon CHU, Seoul (KR); Jae-Kyu ROH, Seoul (KR); Manho KIM, Seoul (KR); Sang Kun LEE, Seoul (KR); Keun-Hwa JUNG, Seoul (KR); Soon-Tae LEE, Seoul (KR); Daejong JEON, Daejeon (KR); Wooseok IM, Seoul (KR); Jae-Jun BAN, Seoul (KR)

(73) Assignee: SNU R&DB FOUNDATION, Seoul (KR)

(21) Appl. No.: 14/604,083

(22) Filed: Jan. 23, 2015

Related U.S. Application Data
(60) Provisional application No. 61/931,866, filed on Jan. 27, 2014.

(51) Int. Cl. A61K 35/28; A61K 38/02

(52) U.S. Cl. CPC A61K 35/28; A61K 38/02

(57) ABSTRACT

The present disclosure provides adipose cell extracts and its use. The composition comprising the present extracts and method can be used advantageously for a stem cell-based, noninvasive therapy for treating neurologic disease such as stroke and HD. Also, in contrast to the stem cell transplantation, the present method or composition enables the repeated treatments, due to the convenient administration, thus leading to a more efficient prevention and/or curing of the disease of interest.

### CROSS-REFERENCE TO RELATED APPLICATIONS

This application claims the benefit of U.S. Provisional Application No. 61/931,866 filed Jan. 27, 2014 in USPTO, disclosure of which is incorporated herein by reference.

### STATEMENT OF SEQUENCE LISTING

The Sequence Listing submitted in text format (.txt) filed on Jan. 23, 2015, named “SequenceListing.txt”, created on Jan. 20, 2015 (1.08 KB), is incorporated herein by reference.

### BACKGROUND OF INVENTION

1. Field of the Invention

The present disclosure generally relates to compositions and methods to treat neurologic disease using adipose stem cell extracts.

2. Description of the Related Art

There is an increased incidence rate of neurodegenerative diseases such as Parkinson's disease, amyotrophic lateral sclerosis, multiple sclerosis, Huntington's disease, Alzheimer's disease, diabetic retinopathy, multiple infarct dementia and disciform macular degeneration as the human lifespan is extended. Currently, it is estimated that 24 million people are suffered from the disease worldwide. In addition, other types of neurodegenerative diseases such as stroke or the diseases caused by other trauma or damages are on the rise. In 2003, in the Republic of Korea, the number of patients aged 65 and over who are suffering from the brain disease such as dementia, stroke and the like and thus need a medical care/treatment is estimated to be about 620,000 people, which requires 3 trillion 400 billion Korean Won (KRW) as the annual cost of care. Arithmetically, the cost will reach about 4 trillion 800 billion KRW in 2015 and 9 trillion KRW in 2030 as the population of the elderly is increased to 870,000 and 1,640,000 people respectively.

Thus there are needs to develop therapeutic agents for neuroprotection to treat and/or prevent the damages or death of nerve cells resulted from the acute or chronic neurodegenerative diseases. However effective therapies have yet to be developed.

Recently the use of stem cells to treat neurodegenerative disease has become of interest. However, their use for the treatment is limited by the difficulty in securing sufficient amounts of autologous stem cells required for the treatment in addition to the low rate of differentiation into the cells with desired function and the low survival rate after the transplantation. Particularly, adipose tissues represent an abundant and accessible source of adult stem cells. But the lack of suitable means to transplant them into nervous system as well as the fact that they are not fully differentiated into nerve cells limits their clinical applications. Also it is further hampered by the fact that the direct transplantation of adipose stem cells into brain requires a brain surgery and only 13% of stem cells transplanted is survived in one month after the transplantation, and almost none in several months after the transplantation (Lee et al., Ann Neurol. 2009 November; 66(5):671-81).

WO 2011/054100 relates to stem cell extracts and uses thereof for immune modulation and discloses compositions and methods for modulating a subject's immune response and inducing immune tolerance using extracts of stem cells such as human embryonic stem cells.

US Patent Application Publication No 2010/0184225 relates to use of a cellular extract for a mitotic remodeling of chromosomes and discloses female germinal cell (egg) extract of pluricellular organisms in M-phase of the cell cycle for a mitotic remodeling of chromosomes of donor cells of pluricellular organisms.

Therefore, there exist needs to develop an alternative neuro-protective medicine which are safer and easy to be administered enabling the repeated treatments.

### SUMMARY OF THE INVENTION

The present disclosure is to provide a therapeutic composition and method to treat neurologic disease.

In one aspect, the present disclosure provides a method of treating or preventing neurologic disease comprising administering an effective amount of adipose stem cell extract or the composition comprising the same to a subject in need thereof.

In one embodiment, the adipose stem cell extract activates pCREB-PGC-1alpha pathway, regulates inflammatory response or suppresses neuronal cell-death.

In other embodiment, the adipose stem cell from which the present extract is prepared is autologous or allogenic or heterologous in nature.

In one embodiment, the present extract includes at least one of a soluble and/or an insoluble matter or material wherein the soluble material comprises a protein, polypeptide or peptide by which the therapeutic effect is exhibited.

The present extract having neuroprotection effects can be advantageously used for treating neurologic disease including an acute neurodegenerative disease, an subacute neurodegenerative disease and a chronic neurodegenerative disease.

In one embodiment, the present extract is prepared by a process which comprises providing an adipose tissue stem cell; preparing a cell suspension of the adipose tissue stem cell by lysing, disrupting or permeabilizing the stem cell; and fractionating the cell suspension into a soluble and an insoluble fraction.

In other embodiment, the suspension is prepared by the lysis wherein the lysis is performed by sonification and the stem cell is pretreated with a proteinase such as collagenase and washed with a buffer before the lysis.

Also provided in the present disclosure is a method for activating pCREB-PGC-1alpha pathway in vivo and/or in vitro comprising administering an effective amount of adipose stem cell extract or the composition comprising the same to a subject in need thereof or cells.

Also provided in the present disclosure is a method for regulating an inflammatory response comprising administering an effective amount of adipose stem cell extract or the composition comprising the same to a subject in need thereof or cells.

Also provided in the present disclosure is a method for suppressing neuronal cell-death comprising administering an effective amount of adipose stem cell extract or the composition comprising the same to a subject in need thereof or cells.

Also provided in the present disclosure is a composition which comprises an adipose stem cell extract and a pharmaceutically acceptable carrier.

In one embodiment, the present composition activates pCREB-PGC-1alpha pathway, regulates inflammatory response and/or suppresses of neuronal cell-death.

The present extract or the composition having neuroprotection effects can be advantageously used for treating neurologic disease comprising an acute neurodegenerative disease, an subacute neurodegenerative disease and a chronic neurodegenerative disease.

The foregoing summary is illustrative only and is not intended to be in any way limiting. Additional aspects and/or advantages of the invention will be set forth in part in the description which follows and, in part, will be obvious from the description, or may be learned by practice of the invention.

### BRIEF DESCRIPTION OF THE DRAWINGS

These and/or other aspects and advantages of the invention will become apparent and more readily appreciated from the following description of the embodiments, taken in conjunction with the accompanying drawings of which:

FIG. 1A to 1D shows a protection of Neuro2A cells against in vitro OGD (oxygen and glucose deprivation) by pre-treatment with hASCs-E (cell free extract of human adipose stem cells).

FIG. 1A shows the morphology of the cells treated with various materials observed under microscope.

FIGS. 1B and 1C are the results from WST-1 and LDH assays, respectively using cells treated with various materials as indicated in the figures and show that the cells treated with hASCs-E significantly increased cell viability (B, WST-1 assay) and attenuated the cytotoxic effect under OGD (C, LDH assay). FIG. 1D shows the concurrent treatment of hASCs-E with OGD also increased the cell viability (D, WST-1 assay). Further it shows that when hASCs-E in which protein has been inactivated by heat treatment containing DNA, RNA or lipid alone had no protection activity. *P<0.05 and **P<0.01, ANOVA. Cell viabilities were normalized to non-OGD controls (B′-D).

FIG. 2A to 2C shows the neuroprotective effects of hASCs-E on various in vivo ischemic stroke models and each are representative photographs of (A) TTC staining and the evaluation of ischemic volumes 1 day after permanent; (B) 90-min transient; and (C) MCA occlusion or permanent Tamura MCA electrocoagulation, showing reduced ischemic sizes in the hASCs-E treatment, which indicates a protective effect of the hASCs-E compared to the controls. *P<0.05, ANOVA. #P<0.05 (between the fibroblast-E and the vehicle-treated control), Student's t-test. Scale bar indicates 0.8 cm.

FIG. 3A to 3C shows the functional and neurological improvements in the ICH model treated with hASCs-E.

FIG. 3A shows that brain water content was significantly lower in the ipsilateral (hemorrhagic) hemispheres of the hASCs-E-treated group than those in the control and fibroblast-E groups 3 days after inducing ICH (**P<0.01, ANOVA).

FIG. 3B is a representative photomicrographs of hemorrhagic brain slices and an evaluation of hemorrhage volumes showed the protective effect of the hASCs-E treatment (*P<0.05, Student's t-test). Scale bar indicates 0.8 cm.

FIG. 3C is the result from one and three days after ICH insult, the hASCs-E group exhibited less profound behavioral deficits on the MLPT test than did the other groups. **P<0.01, ANOVA.

FIG. 4A is a schematic representation of an experimental design used for the present disclosure showing schedule for ASCs-E injection, behavior test, weight measure brain sampling.

FIG. 4B is a result showing that ASCs-E injection mitigated weight loss in R6/2 mouse at 12 weeks age.

FIG. 4C is a result showing that rotarod test showed better motor performance at 10, 11 and 12 weeks of age in ASCs-E treated R6/2 compared with control. * P<0.05, ** P<0.01.

FIG. 5A is a result showing that Striatal mHtt aggregation was mitigated in ASCs-E treated group.

FIG. 5B is a result of western blot showing that mHtt aggregation.

FIG. 5C is a result showing that mHtt aggregation level was higher in ASCs-E treated R6/2 mouse brain compared with R6/2 control.

FIG. 5D is images of the striatum section stained with Niss1 in R6/2 mouse treated with ASCs-E or vehicle.

FIG. 5E is a result showing that striatum/peristriatum ratio is higher in ASCs-E injected group compared with vehicle treated group. Bar=100 mm, * P<0.01.

FIGS. 6A and 6B shows the restoration of p-Akt, CREB and PGC-1a by ASCs-E in which bar graphs show the relative levels of protein expressions normalized to β-actin. * P<0.05.

FIG. 6A is a result of the western blot analysis showing the upregulation of PGC-1a, p-CREB and p-Akt in ASCs-E treated R6/2 mice brain.

FIG. 6B is a result of the western blot analysis showing the upregulation of PGC-1a and p-CREB in ASCs-E treated neuro2A cells.

FIG. 7 is a diagram of DNA expression microarray with the brain of rats treated with hASCs-E after permanent MCA occlusion in the present disclosure. The numbers in the circles indicate the numbers of genes showing a change and a statistical difference (P<0.05, Mann-Whitney U test) in the level of the expression (N, normal; C, control; A, hASCs-E). C/N or A/C indicates the ratio of C:N or A:C, respectively. “≧” or “≦” means a more or less than 2-fold change in the ratio, respectively.

### DETAILED DESCRIPTION OF THE EMBODIMENTS

The present disclosure is based on the discovery that the adipose stem cell extract can be used to treat neurologic diseases and the identification of their mechanism of action.

Thus in one aspect, the present disclosure relates to a method of treating or preventing neurologic disease comprising administering an effective amount of adipose stem cell extract to a subject in need thereof.

Further the present disclosure provides a composition comprising an adipose stem cell extract and a pharmaceutically acceptable carrier for treating or preventing neurologic disease.

So far there has been no report that the adipose stem cell extracts can be used to treat neurologic disease.

The term “Adipose derived stem cell or adipose stem cell (ASC) as used herein refers to isolated multipotent cells which are plastic adherent. The example of which includes but is not limited to ADS (adipose stem cell), ADASC (adipose-derived adult stromal cell), ADSC (adipose-derived stromal cell), ASC (adipose stromal cell), AdMSC (adipose mesenchymal stem cells), lipoblast, pericyte, preadipocyte and PLA (processed lipoaspirate). ASCs have a form which is similar to fibroblast and express various CD markers such as CD10, CD13, CD29, CD34, CD44, CD54, CD71, CD90, CD105, CD106, CD117. Also ASCs can be cultured and multiplied in vitro and differentiated into various cell types. The ASCs according to the present disclosure include cells obtained directly from the subject, cells that are cultured in vitro and recombinant cells that are genetically modified for a desired function for example to express a particular protein of interest.

The ASCs which may be used for preparing the present extract can be isolated from mammals such as rats, mice, cats, dogs, cows, pigs, rabbits, and primates. Exemplary primates include monkeys, chimpanzees, and humans. The ASCs may be isolated using the methods known in the art. For example, ASCs can be isolated from the liposuction product or subcutaneous adipose tissues and the isolated cells can be cultured and maintained in vitro. For the isolation and culture of ASCs, Rodriguez A M, et al., Biochimie 87(1):125-128, 2005; Zannettino A C, et al., J Cell Physiol 214(2):413-421, 2008; and Traktuev D O, et al., Circ Res 102(1):77-85, 2008 may be referred.

The ASCs which may be used for the present extract or the composition comprising the same may be of autologous, allogenic or a heterologous origin.

The term “extract” as used herein refers to a material, particularly cell free, having an effect according to the present disclosure and that comprises a biological material of natural or recombinant origin such as proteins, nucleic acids and/or lipids. The extract can be prepared using all or part of ASCs by lysis, rupturing, permeabilization, and/or poration thereof.

The present extract comprises soluble and/or insoluble matter or materials which can be separated into a soluble and insoluble fraction. In one embodiment, a soluble matter, material or fraction is used. Contained in the soluble fraction is at least one of a protein, a polypeptide or a peptide, DNA, RNA and/or lipids. In one embodiment, soluble fraction contains a protein, a polypeptide or a peptide. In other embodiment, the insoluble fraction contains lipids. As described in hereinafter, it was found in the present disclosure that the neuroprotective effect is exerted or exhibited by the proteins contained in the soluble fraction.

The lysis, rupturing, permeabilization, and/or poration of ASCs may be performed using chemical and/or physical methods, such as for example freezing and thawing, sonification, treatment with enzymes including such as hyaluronidase, dispase, collagenase, nucleases and/or chemical treatment using such as DTT (Dithiothreitol). For example Taranger C K et al., Mol Biol Cell 16(12):5719-35, 2005; Cho H J et al., Blood 116(3):386-95, 2010; Lee S T, et al., Plos One 6(7):e21801, 2011; and Jeon D, et al., Epilepsia. 52(9):1617-26, 2011 may be referred.

It was found in this disclosure that the present ASCs extract or the composition comprising the same provides a direct neuroprotection effect or delays the progression of the neurologic diseases by regulating the pathologic mechanism thereof.

Thus the present extract or the composition comprising the same can be advantageously used for treating and/or preventing various neurologic diseases in which the neuroprotection is desired or the regulation of the pathologic mechanism is desired. Such diseases include, but are not limited to an acute neurodegenerative disease, a subacute neurodegenerative disease and a chronic neurodegenerative disease.

The term “neurologic disease, disorder, condition or symptom” as used herein refers to a disease, disorder, condition or symptom that affects the brain, spinal cord and peripheral nerves and that are accompanied by paralysis, incontinentia, parakinesia, cognitive dysfunction, clouded consciousness and/or spasm seizure and the like due to the structural, chemical and/or electrical dysfunction of the nerve system.

The term “neuroprotection or neuroprotective” as used herein refers to the protection of nerve cells in the brain, central nerve system, or peripheral nerve system (particularly in the brain and spinal cord) from neuronal cell-death and/or possible damages. Also encompassed by that term is treating neurologic diseases, preventing and/or delaying the onset and/or progression of the diseases of interest.

Also, the neuroprotection in the present disclosure encompasses the protection of the brain, central nerve system, or peripheral nerve system which already has been damaged. Herein the damages include but are not limited to ones caused by chemicals, toxic chemicals, inflammations, radiation and/or traumatism.

Without being bound by the theory, in one embodiment, the present extract or the composition comprising the same exerts or exhibits its function or effect as described above by activating pCREB-PGC-1α pathway. Thus, the present extract or the composition comprising the same can be advantageously used for treating and/or preventing various diseases in which the activation of pCREB-PGC-1α pathway is required. Further, in other embodiment, the present extract or the composition regulates inflammatory response as clearly shown in Table 1 for example, or suppresses neuronal cell-death as shown by, for example, table 1 and the neuroprotective effect.

In the present disclosure, the acute neurodegenerative disease includes various diseases associated with dead or damaged nerve cells due to insufficient cerebral blood flow, local brain trauma, or brain or spinal cord injuries. For example, it includes but is not limited to embolic occlusion and thrombotic occlusion, acute ischemia, perinatal hypoxic ischemic damage, reperfusion after intracranial hemorrhage (for example including epidural, subdural, subarachnoid and intracerebral) and cardiac arrest, cerebral ischemia by intracranial and intraspinal lesion (for example bruises, penetrating injury, compression and laceration) or cerebral infarction. In one embodiment, the acute neurodegenerative disease includes stroke, cerebral infarction, cerebral hemorrhage, head injury and spinal cord injury.

In the present disclosure, the subacute neurodegenerative disease refers to a neurologic disease developing over a period time from a few days to weeks and includes, but is not limited to demyelinating disease including multiple sclerosis, paraneoplastic neurological syndrome, subacute combined degeneration, subacute necrotizing encephalitis, or a subacute sclerosing encephaliti.

In the present disclosure, the chronic neurodegenerative disease includes but is not limited to a senile dementia, a vascular dementia, a diffuse white matter disease (binswanger's disease), a dementia of endocrine or metabolic origin, a dementia due to head Injury or diffuse brain damage, an amnesia including dementia pugilistica and frontal lobe dementia, an Alzheimer's disease, a pick disease, a diffuse lewy body disease, a progressive supranuclear palsy (steele-richardson-olszewksi syndrome), a multiple system degeneration (shy-drager syndrome), a neurodegeneration related to Chronic epilepsy syndrome, an amyotrophic lateral sclerosis, a motor incoordination, a corticobasal degeneration, an Amyotrophic lateral sclerosis-parkinsonism/dementia complex of Guam, a subacute sclerosing encephalitis, a Huntington's disease, a Parkinson's disease, a synucleinopathy, a primary progressive aphasia, a striatonigral degeneration, a Machado-Joseph disease/spinocerebellar ataxia, a motor neuron cell disease including an olivopontocerebellar degeneration, a Gilles de la tourette disease, a bulbar and pseudobulbar paralysis, a spinal cord and spinobulbar amyotrophy (Kennedy Disease), a multiple sclerosis, a primary lateral sclerosis, a hereditary spastic paraplegia, a Werdnig-Hoffman disease, a Kugelberg-Welander disease, a tay-sachs disease, a sandhoff disease, a hereditary spastic disease, a Wohlfart-Kugelberg-Welander disease, a spastic paraplegia, a progressive multifocal leukoencephalopathy, a hereditary autonomic dysfunction (riley-day syndrome), a creutzfeldt-jakob disease, a gerstmann-strauissler-scheinker disease, a prion disease including a kuru disease and a fatal familial insomnia, or a cerebral palsy.

The present extract or the composition comprising the same provides the neuroprotection activity against the nerve damages or injuries associated with the neurologic diseases as described above. Thus the present extract or the composition comprising the same can be used for treating or preventing neurologic disease by administering an therapeutically effective amount of adipose stem cell extract or the composition comprising the same to a subject in need thereof

As used herein, the phrase “therapeutically effective amount” when used in connection with adipose stem cell extract or the composition comprising the same means an amount of thereof effective for treating, attenuating, reducing the severity of a disease or disorder disclosed herein, reducing the duration of a disease or disorder disclosed herein, prevent the advancement of a disease or disorder disclosed herein, one or more symptoms associated with a disease or disorder disclosed herein.

As used herein, the terms “treat,” “treatment,” and “treating” include alleviating, abating or ameliorating at least one symptom of a disease or condition, and/or reducing severity, progression and/or duration thereof, and/or preventing additional symptoms, and includes prophylactic and/or therapeutic measures.

The present extract or the composition comprising the same may be used alone or in combination with other treatment such as surgery, or other therapeutic agents.

The composition of the present disclosure further includes at least one pharmaceutically acceptable carries or excipient in addition to the active ingredient.

The carriers or excipient which may be used for the present pharmaceutical composition are materials which are not toxic or harmful to the cells, tissues or organs to be treated and include but are not limited to, saline, sterilized water, Ringer's solution, buffered saline, phosphate, citrate and other organic buffers, antioxidants such as ascorbic acids, polypeptide less than 10 amino acid in length, proteins such as serum albumin, gelatin or immunoglobulins, hydrophilic polymers such as polyvinylpyrrolidone, amino acids such as glycine, glutamine, asparagine, arginine or lysine, monosaccharides, disaccharides and glucose, or other carbohydrates such as mannose, chelating agents such as EDTA, sugar alcohols such as mannitol or sorbitol, salt forming counterion such as Na, and/or nonionic surfactant such as Tween, polyethylene glycol and PLURONICS,

If desired, the composition may further include antioxidant, buffer, antibacterial agents, and other additives known in the art to prepare pharmaceutical compositions. The present composition may further include lubricants, wetting agents, sweetening agents, flavors, emulsifier, suspending agents to be formulated for example as a unit dosage form such as tablets, capsules or gel (such as hydrogel), which may further include dispersion agents or stabilizers. Further the latest edition of Remington's Pharmaceutical Science (Mack Publishing Company, Easton Pa.) may be referred for the preparation and formulation of pharmaceutical composition.

The present extract and composition can be administered by various routes known in the art such as oral or parenteral delivery for example intravenous, subcutaneous, or intraperitoneal injections or delivery through patch, nasal or respiratory patches. In one embodiment, injections are preferred. In particular, parenteral deliveries are preferred.

Desirable or optimal dosage may vary among patients depending on various factors such as body weight, age, sex, general condition of health, diet, severity of diseases, and excretion rate. The typical unit dosage includes but does not limit to for example about 0.01 mg to 100 mg a day. Typical daily dosage ranges from about 1 ng to 10 g and may be administered one or multiple times a day.

In one embodiment, the extract is provided by a process which comprises providing a adipose tissue stem cell; and preparing a cell suspension of the adipose tissue stem cell by lysing, disrupting or permeabilizing the stem cell.

Thus, also embodied in the present disclosure is a method for preparing ASCs extract, which comprises a) providing adipose tissue stem cells; and b) preparing a cell suspension of the adipose tissue stem cell by lysing, disrupting or permeabilizing the stem cells.

In one embodiment, the process further comprises fractionating the cell suspension into a soluble and an insoluble fraction.

In other embodiment of the process, the adipose stem cells are lysed. In still other embodiment, the adipose stem cells are treated with collagenase followed by washing with a buffer such as phosphate buffered saline and then lysed by sonification.

In one embodiment, the method further comprises before the step b), the step of pretreating the adipose stem cells with collagenase followed by washing with a buffer such as phosphate buffered saline.

Alternatively, instead of the step b), the adipose stem cells are pretreated with collagenase followed by washing with a buffer such as phosphate buffered saline and then lysed by sonification.

In other aspect, the present disclosure provides a method of treating or preventing neurologic disease comprising administering an effective amount of adipose stem cell extract or the composition comprising the same to a subject in need thereof.

As described herein above, the present extract or the composition comprising the same exhibits its neuroprotective effect or function through the activation of pCREB-PGC-1alpha pathway, the regulation of inflammatory response or the suppression of neuronal cell-death.

In this aspect, the present disclosure further provides a method for activating pCREB-PGC-1alpha pathway in vitro or in vivo comprising administering an effective amount of adipose stem cell extract or the composition comprising the same to a subject in need thereof or cells.

In other aspect, the present disclosure further provides a method for regulating an inflammatory response in vitro or in vivo comprising administering an effective amount of adipose stem cell extract or the composition comprising the same to a subject in need thereof or cells.

In other aspect, the present disclosure further provides a method for suppressing neuronal cell-death in vitro or in vivo comprising administering an effective amount of adipose stem cell extract or the composition comprising the same to a subject in need thereof or cells.

Regarding the ASCs extract, the composition, and the types of diseases to be treated, the descriptions hereinbefore may be referred.

The present disclosure is further explained in more detail with reference to the following examples. These examples, however, should not be interpreted as limiting the scope of the present invention in any manner.

### EXAMPLES

### Materials and Methods

1. Preparation of Extract of Human Adipose Tissue and Human Fibroblast

Subcutaneous adipose samples were obtained from a normal human who provides the written informed consent to participate in this study and Institutional Review Board in Seoul National University Hospital approved this study.

To obtain stem cells from the human adipose tissues, the human adipose tissues were minced and treated with a PBS solution comprising 0.075% collagenase type I (Invitrogen, USA) at 37° C. for 1 hour with gentile agitation. Then the digested tissues ware centrifuged at 200×g for 10 min and the upper adipocyte fraction was removed from the lower stromal fraction (pellet) which was then treated for 10 min with red blood cell lysis buffer (Sigma, USA) at RT and filtered through a 100-mm nylon mesh, and centrifuged at 200×g for 10 minutes. The filtrate was then suspended in endothelial cell growth medium 2MV (EGM2 MV; Lonza, USA) and incubated therein

The hASCs or fibroblast extracts were prepared as follows. The cultured ASCs or fibroblasts were harvested, washed twice with phosphate-buffered saline (PBS), and centrifuged at 200×g for 5 min. The ASCs and fibroblasts were suspended in buffer (1 mM DTT, 1 mM EDTA, protease inhibitor cocktail P8340, 0.1% DEPC in PBS) and lysed within a syringe tube with a plugged end by pushing and pulling the syringe piston several times. Cell lysates were centrifuged at 14,000×g for 15 min, and the final extract was produced by passing the supernatant through a 0.45 μm syringe filter unit using a 10 mL syringe. About 5×10⁶ cells were used to make 100 mg of cell extracts. As a extraction buffer for in vitro treatment, DMEM (Dulbeccos modified Eagles medium, Welgene, Korea) was used. The ASCs-E and fibroblast-E were freshly prepared just before intraperitoneal (i.p.) administration.

Human fibroblast are cultured as described previously (Jeon, D., et al. 2011. Epilepsia 52, 1617-1626).

2. Culture of Neuro-2a Cells and its Treatment with hASC-E

Neuroblast (Neuro-2a, American Type Culture Collection, USA) was cultured in DMEM supplemented with 10% fetal bovine serum, 100 U penicillin, and 0.1 mg/mL streptomycin (Gibco, Grand Island, N.Y., USA) at 37° C. in 5% CO₂/95% air.

Two days after the seeding, Neuro-2a cells were treated with 10 μg/ml of hASC-E for 24 hrs. Then cells were harvested and lysed in RIPA (Radio Immuno Precipitation Assay) buffer supplemented with protease and phosphatase inhibitors (Roche, USA). After cellular debris was cleared by centrifugation, protein concentration was determined by the BCA protein assay (Pierce, USA) according to the manufacturer's instructions.

3. In Vitro Ischemia and Cell Viability Assays

In vitro ischemia and cell viability assays were performed as described previously (Jung et al., 2006 Neurosci. Lett. 394, 168-173; Park et al., 2009 Neurosci. Lett. 451, 1619). Briefly, mouse neuroblast cells (Neuro-2a, American Type Culture Collection, Manassas, Va., USA) were plated at a density of 5×10³ cells/well in 96-well plates and grown for 24 h in Dulbecco's modified Eagle's medium (Welgene, Daegu-si. South Korea) supplemented with 10% fetal bovine serum, 100 U penicillin, and 0.1 mg/mL streptomycin (Gibco, Grand Island, N.Y., USA) at 37° C. in 5% CO₂/95% air. On day 2, the media were replaced by media mixed with hASCs-E (100 μg/well), and the cells were grown for 24 h under the same conditions as described above. For comparison or controls, PBS, heat-treated hASCs-E, DNA,RNA, or lipid were used instead of hASCs-E. On day 3, in vitro ischemia was induced by oxygen glucose deprivation (OGD, no glucose and sodium pyruvate, O₂ levels 1-1.5%) for 16 h in a humidified hypoxic chamber (Bactron 1.5, Sheldon Manufacturing, Cornelius, Oreg., USA). DNA, RNA, lipids, and heat-treated hASCs-E were prepared from the same amount of hASCs that were used to obtain 100 μg hASCs-E. The hASCs-E was boiled for 10 min for the heat treatment.

For concurrent treatment of hASC-E with OGD, hASC-E (100 μg/well) or PBS was applied to Neuro-2a cells and in vitro OGD was induced immediately.

Cell viability and cytotoxicity were quantified with the WST-1 reagent (5 mmol/L, 1:9, Roche, Mannheim, Germany) and lactate dehydrogenase (LDH, CytoTox 96, Promega, Madison, Wis., USA) assays according to the manufacturer's instruction. WST-1 or LDH activity is based on the enzymatic conversion of the WST-1 reagent or a tetrazolium salt into a red formazan product by dehydrogenases or diaphorases, respectively. Absorbance was measured at 440 nm (WST-1 assay, 60 min incubation) or 490 nm (LDH assay, 30 min incubation) using a SpectraMax190 instrument (Molecular Devices, Eugene, Oreg., USA). All in vitro ischemia experiments were triplicated. In the WST-1 assay, a total of 21 wells were used for PBS, 12 wells for hASCs-E, 12 wells for heat-treated hASCs-E, 11 wells for DNA, 12 wells for RNA, and 11 wells for lipid. In the LDH assay, a total of 24 wells were used for PBS, 12 wells for hASCs-E, 18 wells for heat-treated hASCs-E, 12 wells for DNA, 11 wells for RNA, and 12 wells for lipid. Data are normalized as the ratio of each non-OGD value.

4. Generation of Experimental Stroke Models and Treatment of hASCs-E

Experimental stroke models (transient or permanent MCA occlusion, and focal cerebral ischemia and ICH) were generated using Male Sprague-Dawley rats (weight, 240-260 g, Orient Bio, Republic of Korea) and male BALB/c mice (weight, 22-25 g, Orient Bio) as described previously (Chu, K. et al., 2007, Stroke 38, 177-182; Lee, S. T., 2008. Brain 131, 616629). Animal care and handling were carried out according to the guidelines of the Institutional Animal Care and Use Committee of Seoul National University Hospital. The hASCs-E (40 mg/kg to rats, i.p.) and control vehicle (equal volume of a lysis buffer instead of extracts), or fibroblast-E (40 mg/kg to rats, i.p.) were administered 1 h after stroke insults. Treatment (40 mg/kg to mice, i.p.) was administered 3 h after the induction of ischemia in the focal cortical infarction mouse model induced by electrocoagulation, because of the time required for the procedure. The doses of extracts were determined according to a previous report (Jeon, D et al., 2011, Epilepsia 52, 1617-1626). Physiological parameters, including mean arterial blood pressure, blood gases, and glucose concentration, were measured throughout all stroke model surgeries. Rectal temperature was maintained at 37±0.5° C. using a thermistor-controlled heating blanket.

5. Measurement of Ischemic Volume

At 24 h after the ischemic insults, ischemic volume (infarcted tissue and penumbra) was evaluated in the ischemia model using 2,3,7-triphenyltetrazolium chloride (TTC) staining, as described previously (Chu, K. 2007. Stroke 38, 177-182). Briefly, the brain was removed, and cut from the frontal tip by 1 mm thickness, which was then immersed in a 2% solution of TTC. Stained slices were fixed in phosphate-buffered 4% paraformaldehyde, and the ischemic and total hemispheric areas of each section were traced and measured using an image analysis system (Image-Pro Plus, Media Cybernetics, Silver Spring, Md., USA). The corrected ischemic volume was calculated to compensate for the effect of brain edema, as follows: corrected ischemic area=measured ischemic area×{1−[(ipsilateral hemisphere area−contralateral hemisphere area)/contralateral hemisphere]}. Ischemic volumes were determined by two independent investigators blinded to the type of section, and were expressed as percentages of total hemispheric volumes.

6. Measurement of Brain Water Content and Hemorrhage Volume in the ICH Model

Brain water content and hemorrhage volume were measured 3 days after ICH, as described previously (Lee, S. T. 2008. Brain 131, 616-629). Briefly, the brains of rats were removed immediately after anesthetization followed by decapitation to measure water content. After the cerebellum was removed, the brain was divided into two hemispheres along the midline. The two hemispheres were immediately weighed on an electronic analytical balance to obtain wet weights, and they were dried in a gravity oven at 100° C. for 24 h to measure dry weight. Water content was expressed as a percentage of wet weight using the following formula: ([wet weight−dry weight]/wet weight)×100 (%).

Drabkin's reagent (Sigma) was used to measure hemorrhage volumes according to the manufacturer's instruction. ICH hemispheric brains were isolated after transcardiac perfusion to remove blood. Each hemispheric sample was homogenized and sonicated, and then centrifuged at 12,000 rpm for 30 min. The supernatant (0.4 mL) was mixed with Drabkin's reagent (1.6 mL) and allowed to stand for 20 min at room temperature. Optical density was then measured and recorded at 540 nm with a spectrophotometer (Molecular Devices). The values were compared to those of a standard curve, which was acquired from Drabkin's reaction of normal hemispheric brains and homologous blood (0, 2, 4, 8, 16, 32, 50, and 100 μL).

7. Huntington's Disease Animal Model and ASCs-E Injection Schedule

Transgenic HD mice of the R6/2 line (B6CBATg(HDexon1)62Gpb/3J, 111 CAGs) and their WT littermates (Jackson Laboratories, USA) were used. The R6/2 transgenic mouse model expresses exon 1 of a human mHtt and is the most widely used animal model for studying HD. These mice were obtained by crossing ovarian transplant hemizygote females with B6CBAF1/J males. The mice were housed in groups with ad libitum access to food and water and a 12 hours light/12 hours dark cycle. The genotype was assessed using a PCR assay.

In R6/2 mice, disease phenotype appears at 8 weeks of age. Considering protective effect of ASCs-E, injection was started at 6 weeks old. ASCs-E was injected intraperitoneally two times a week (40 mg/kg) and control mice were injected with vehicle (equal volume of extraction buffer). The ASCs-E was freshly prepared just before administration as described before.

8. Rotarod Performance and Weight Measurement

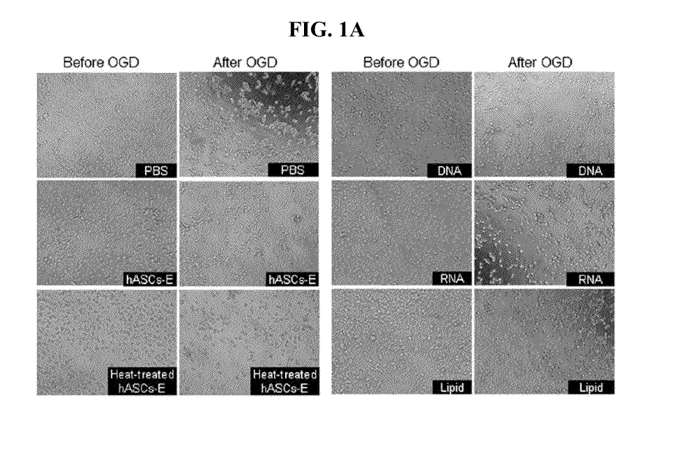

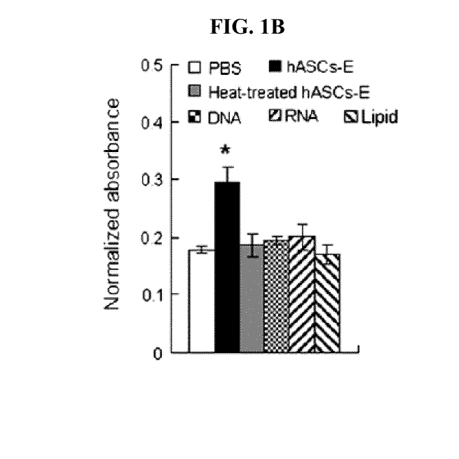

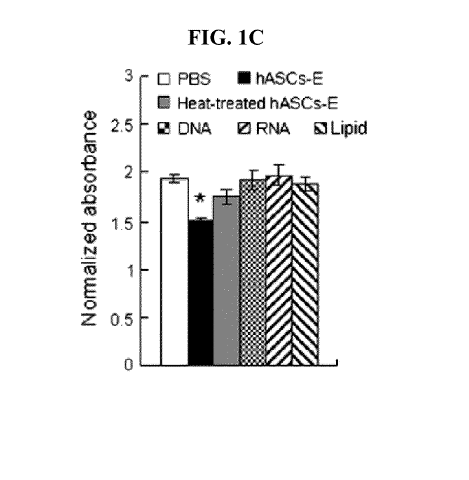

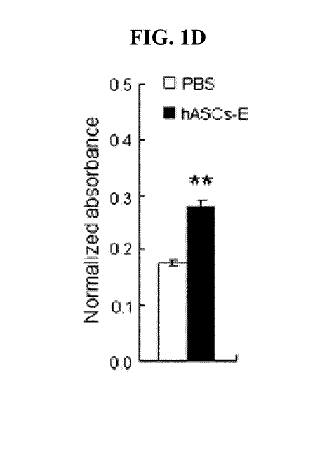

The Rotarod test was evaluated using a rotarod apparatus Jungdo Instruments, Korea). Mice were placed on the rod with an accelerating rotating speed from 4 to 40 rpm over a period of 3 minutes with a 15 minutes rest between trials. Mice were trained on three consecutive days for three trials per day at 4 weeks of age. Three trials were performed and the mean latencies to fall were used to analyze data. Rotarod evaluation and weight measurement were performed every week from 5 to 12 weeks (FIG. 1A).

9. MLPT Test

The modified limb placing test (MLPT) was performed 1 day before, 1 day after, and 3 days after ICH insult as previously described (Chu, K. et al. 2007. Stroke 38, 177-182). In this test, sensorimotor integration of the forelimbs and the hind limbs was assessed by monitoring responses to tactile and proprioceptive stimulations. In brief, the rat is suspended 10 cm above a table, and the stretch of the forelimbs towards the table is observed and evaluated: a normal stretch is scored as 0 points; abnormal flexion is scored as 1 point. Next, the rat is positioned along the edge of the table, and its forelimbs are suspended over the edge, allowing them to move freely. Each forelimb (second task, forelimb; third task, hind limb) is gently pulled down, and retrieval placements are evaluated. Finally, the rat is placed near the edge of the table to assess lateral placement of the forelimb. The three tasks are scored as the following: normal performance, 0 points; delayed (at least 2 s) and/or incomplete performance, 1 point; no performance, 2 points. A total score of 7 points indicates maximal neurological deficit, and a score of 0 points indicates normal performance.

10. Immunohistochemistry and Striatal Volume Analysis

For immunohistochemistry, mice were anesthetized and perfused through the heart with 10 ml of cold saline and 4% paraformaldehyde in 0.1 M PBS at 12 weeks of age. Brains were removed from the skull, cryoprotected in 30% sucrose solution at 4° C., and sectioned 20 urn. Free-floating sections were washed and blocked with normal goat serum, then stained with the EM48 mHtt antibody (1:300; Millipore). On the following day, the sections were washed in PBS with three times and incubated with FITC conjugated anti-rabbit IgG (1:50; Jackson Immuno Research Laboratories) for 2 hours. To examine the volume of striatum in R6/2 mice, serial-cut 200-μm coronal tissue sections from the rostral segment of the neostriatum to the level of the anterior commissure (bregma 1.54 to 0.10 mm) were used for Niss1 staining. The areas of the striatum and the peristriatum were defined from each serial section, and gross volumes were measured with integrating each sectional area using Image-Pro Plus (Media Cybernetics, USA).

11. Protein Extracts and Western Blot Analysis

Brains of R6/2 mice were isolated, immediately frozen on liquid nitrogen, and stored at 270 uC until protein extraction. Cultured N2A cells were washed and harvested in PBS using a cell scraper. Protein extracts were prepared using RIPA buffer (Thermo, USA) with freshly added protease inhibitor and phosphatase inhibitor (Roche, USA). 30 mg protein samples were separated on 10% SDS-PAGE and transferred onto PVDF membrane. The blots were probed with primary antibodies: PGC-1a (1:1000; Santa Cruz, Calif.), p-Akt (1:1000; Cell Signaling), CREB (1:1000; Cell signaling), EM48 (1:200; Millipore), followed by horseradish peroxidase conjugated secondary anti-mouse or rabbit antibody (1:5000; GE Healthcare), and blots were developed using ECL. Western blots were scanned and intensity values were obtained using ImageJ software.

12. Gene Expression Microarray

Rats subjected to permanent focal cerebral ischemia were used for the array. RNA was isolated from the brains (N, normal, without any treatment, n=3; C, control, occlusion and treated with lysate buffer, n=3; A, hASCs-E, occlusion and treated with hASCs-E, n=3) by homogenization with TRIzol® Reagent (Gibco). Genes with altered expression levels were classified and grouped according to function. The data analysis criteria were a more than two-fold change in the ratio of C:N (C/N), a statistical difference between C and N, and a statistical difference between A and C (P<0.05, Mann-Whitney U test). Hybridization was performed using a buffer contained in in situ hybridization kit (Agilent, USA) in a oligo microarray (Agilent, USA). The data analysis was performed using GenePix 4000B scanner (Axon Instruments, USA).

Also performed was gene expression microarray for in vitro Neuro2a models after concurrent treatment of hASC-E (or control medium) and OGD. To reduce the influence of inter-group variability, three RNA samples per group were pooled to generate one microarray samples per group (No-OGD+control, No-OGD+hASC-E, OGD+control, and OGD+hASC-E).

13. RT-PCR Using Brain and Spleen in ICH Model

To examine the anti-inflammatory effects of hASC-E treatment in the ICH model, ICH rats (n=3 per group, PBS or hASC-E treated) were sacrificed at 24 h after the ICH induction. Total RNA was isolated from each brain and spleen using TRIzol® Reagent, and we conducted RT-PCR using the First Strand cDNA Synthesis Kit for RT-PCR (Roche, Indianapolis, Ind., USA). The following primer sets were employed (25 cycles of 95, 58 and 72° C. for 40 s each): TNF-α (tumor necrosis factor-α): 5″-TAC TGA ACT TCG GGG TGA TTG GTC C-3′ (sense) as represented by SEQ ID NO: 1 and 5″-CAG CCT TGT CCC TTG AAG AGA ACC-3″ (antisense) as represented by SEQ ID NO: 2; IL-6 (interleukin-6): 5″-CTT GGG ACT GAT GTT GTT GAC-3′ (sense) as represented by SEQ ID NO: 3, and 5″-TCT GAA TGA CTC TGG CTT TG-3′ (antisense) as represented by SEQ ID NO: 4. Then the amplified products were electrophoresed in an agarose gel and quantified the transcript levels via the analysis of scanned photographs of gels, using appropriate imaging software (ImageJ, NIH, Bethesda, Md., USA). The mRNA expression levels were normalized to those of GAPDH. All values shown in the figures are presented as mean 6

14. Statistical Analysis

All values shown in the figures are presented as mean±standard error. Rotarod performance and weights were analyzed by ANOVA with Fisher's post hoc test at each week of age. Western blot and histological results were analyzed by Student's t-test. A 2-tailed probability value below 0.05 was considered statistically significant. Data were analyzed using SPSS version17.0 (SPSS Inc., USA).

### Example 1

### Enhancement of the Cell Viability by hASC-E

The hASCs-E was applied to cultured Neuro2a cells before OGD to test its neuroprotective effect on ischemia injury as described in the materials and methods section. Results are shown in FIG. 1A to 1D. As shown in FIG. 1A, Sixteen hours after OGD, the cells treated with the hASCs-E (100 μg) showed elongated neurites. However, the control cells treated with heat-treated hASCs-E, DNA, RNA, or lipids derived from equal amounts of hASCs-E were smaller than before the treatment, and appeared to be tightly rounded, aggregated, and shrunken with retracted or lessened dystrophic neurites. Also as shown in FIGS. 1B and 1C, Neuro2a cells that had been treated with the hASCs-E showed increased viability in the WST-1 assay (P<0.0001, FIG. 1B) and LDH assay (P<0.001, FIG. 1C) compared to the control groups (PBS-, heat-treated hASCs-E-, DNA-, RNA-, and lipid-treated cells). In addition, concurrent treatment of hASC-E with OGD also increased cell viability compared to the PBS treatment in the WST-1 assay (FIG. 1D, P<0.01).

These results indicate the neuroprotective effect of the hASCs-E against ischemia in vitro and suggest that the main active or effective factors are hASCs-E proteins, not nucleic acids or lipids.

### Example 2

### The Reduction of Ischemic Volume in a Focal Cerebral Ischemia Model Treated with hASCs-E

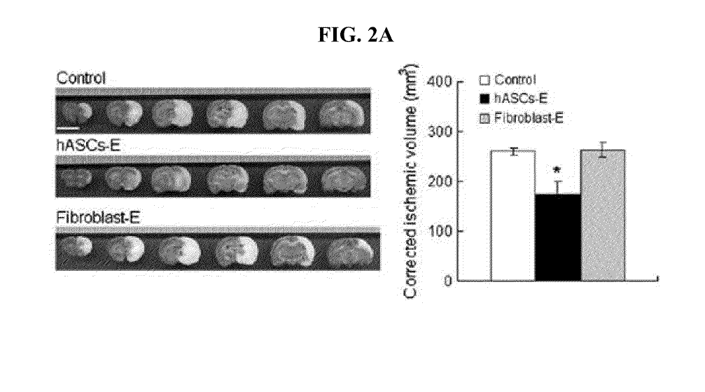

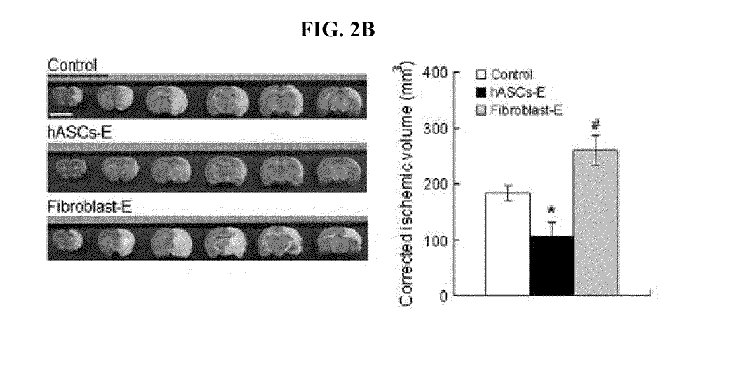

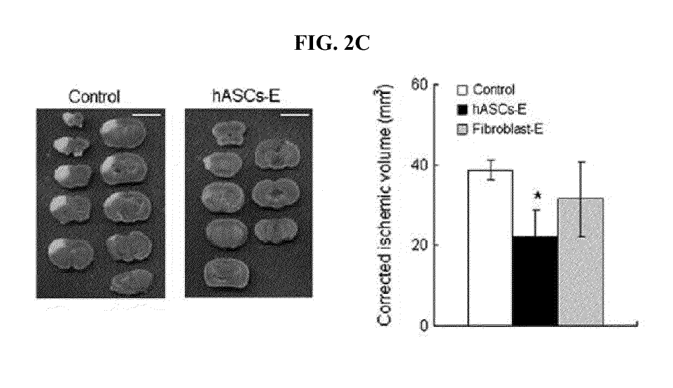

The hASCs-E was systemically applied to animals subjected to permanent or transient MCA occlusions as prepared in the materials and method section to investigate whether hASCs-E affects ischemic neuronal damage in vivo. The results are shown in FIG. 2A to 2C. As shown in FIG. 2A, the rats in the permanent MCA occlusion model treated with hASCs-E 1 h (n=9, 174.02±25.94 mm³) after occlusion showed reduced ischemic volume compared to the control rats treated with PBS (n=11, 261.24±7.03 mm³) (P<0.05; FIG. 2A). Ischemic volume was about 34% smaller in the hASCs-E group than in the control. However, the rats treated with fibroblast-E (n=5, 263.83±13.95 mm³) showed similar ischemic volume levels as the control rats.

As shown in FIG. 2B, rats in the transient MCA occlusion model treated with the hASCs-E 1 h (n=10, 107.44±24.00 mm3, 41% smaller than the control) after occlusion also showed reduced ischemic volumes compared to the control (n=11, 182.48±12.91 mm³) (P<0.05). However, the rats treated with fibroblast-E (n=10, 259.81±26.90 mm³) showed increased ischemic volumes compared to the control rats (P<0.01).

Furthermore, as shown in FIG. 2C, treatment with the hASCs-E 3 h after permanent electrocoagulation in the cortical MCA electrocoagulation model led to reduced ischemic volumes (43% smaller) in mice compared to the controls (hASCs-E, n=9, 21.99±6.62 mm³; control, n=8, 38.72±2.43 mm³, P<0.05). However, the mice treated with fibroblast-E (n=5, 31.43±9.16 mm³) showed no significant difference in ischemic volume compared to the control mice.

### Example 3

### The Amelioration of Neurologic Deterioration and Reduction of Brain Water Content and Hemorrhage Volume in the ICH Model by hASCs-E

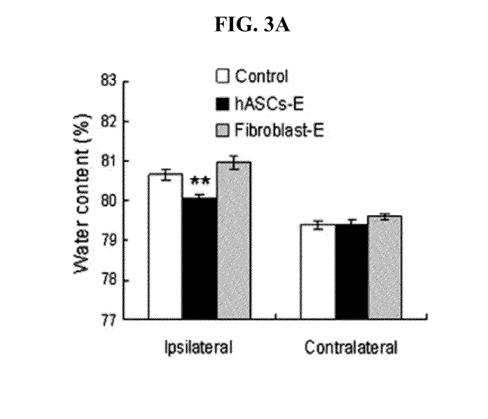

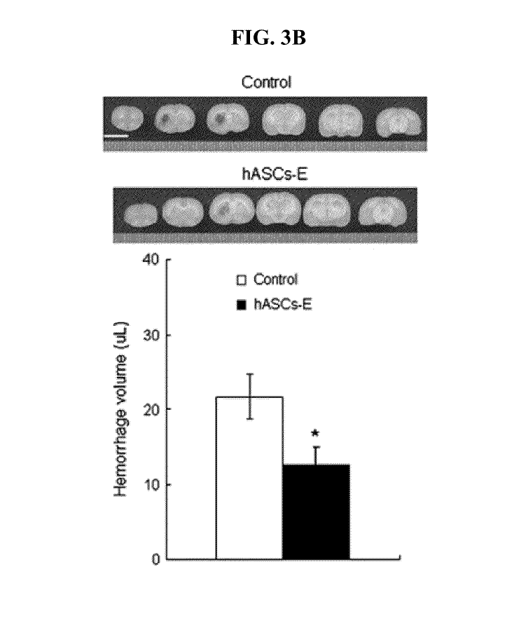

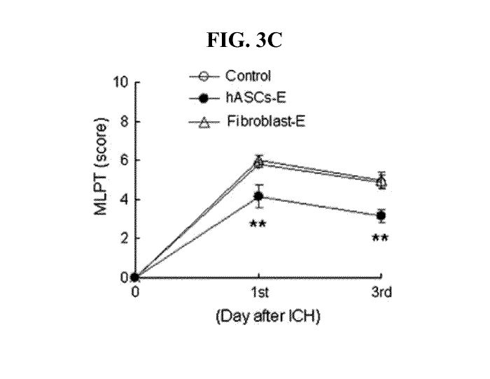

To assess the effect of the hASCs-E in the ICH model, hASCs-E or fibroblast-E was administrated to the ICH model induced by intrastriatal microinjection of bacterial collagenase as described in the materials and method section. Results are shown in FIG. 3 A to 3C. The brain water content measurement is shown in FIG. 3A and hemorrhage volume (FIG. 3B) in brains of ICH rats and also subjected the ICH models to the MLPT test. As shown in FIG. 3A, administering the hASCs-E 1 h after inducing ICH led to decreased water content in ipsilateral (hemorrhagic) hemispheres, compared to the other groups (control, n=11, 80.65±0.14%; hASCs-E, n=6, 80.04±0.11%; fibroblast-E, n=6, 80.96±0.16%) (P<0.01), without any differences in the contralateral (non-hemorrhagic) hemispheres. In contrast, no difference in water content was observed between the control and fibroblast-E groups. Similar to the brain water content results, hASCs-E (n=7, 12.52±2.48) treatment 1 h post-ICH produced reduced hemorrhage volumes compared to the control (n=8, 21.70±3.07) (P<0.05) as shown in FIG. 3B). Also as shown in FIG. 3C, the hASCs-E treatment group showed decreased scores on the MLPT test compared to the other groups (P<0.01, control, n=9; hASCs-E, n=6; fibroblast-E, n=6) at day 1 (control=5.78±0.15, hASC-E=4.17±0.60, fibroblast-E=6.00±0.29) and day 3 (control=4.89±0.35, hASC-E=3.17±0.31, fibroblast-E=5.00±0.37). However, the fibroblast-E group had similar scores as those of the vehicle-treated control ICH group.

These results indicate a favorable neurologic recovery as a result of the hASCs-E.

### Example 4

### Improvement of Behavioral Phenotypes of R6/2 Mice Model by hASCs-E

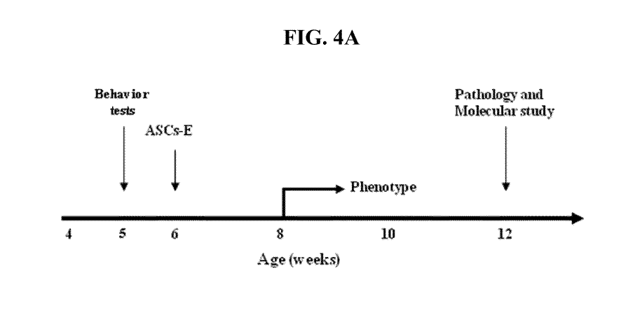

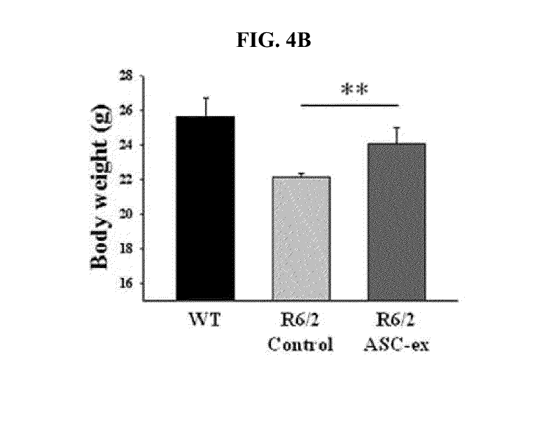

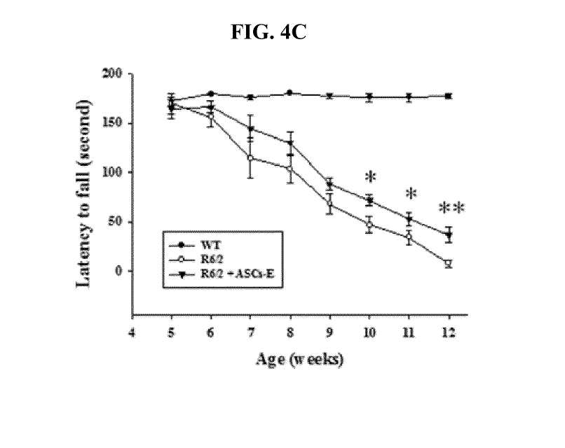

To investigate effects of ASCs-E on behavioral deficits of R6/2 mice, ASCs-E was injected from 6 weeks old and phenotypes were examined Results are shown in FIGS. 4A to 4C. Mice showed gradual weight loss from 10 to 12 weeks of age. In contrast with vehicle treated R6/2 mice, ASCs-E injected R6/2 showed delayed progression of weight loss at 12 weeks old (22.1±0.2 vs. 24±0.9, p<0.01) (FIG. 4B). In rotarod test, ASCs-E treated group showed lowered latency to fall compared with vehicle treated group 10, 11 (p<0.05) and 12 weeks (p<0.01) of age (FIG. 4C).

These results indicate that the present hASCs-E prevent the loss of weight of the Huntington's disease mouse model and improve the motor function.

### Example 5

### Reduction of Striatal Atrophy and Mutant Htt Aggregation of R6/2 Mice by hASCs-E

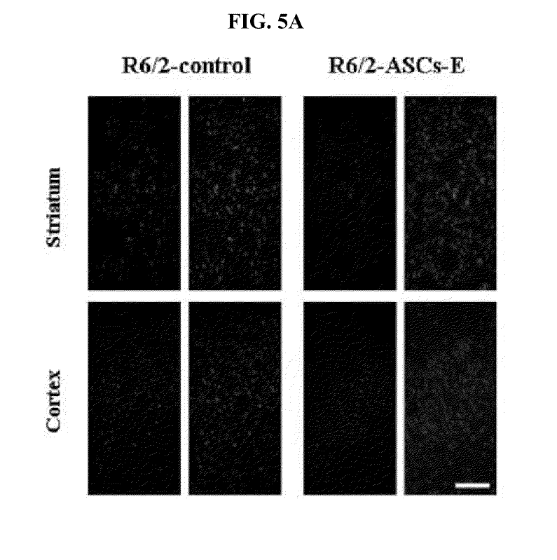

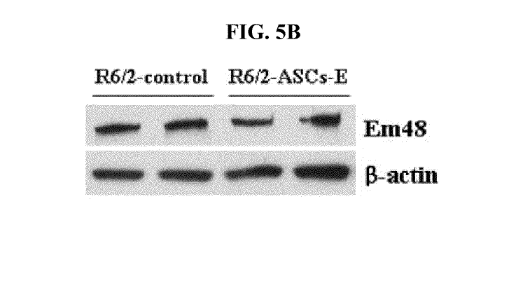

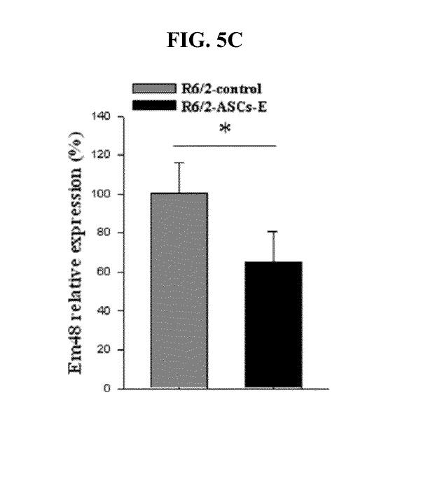

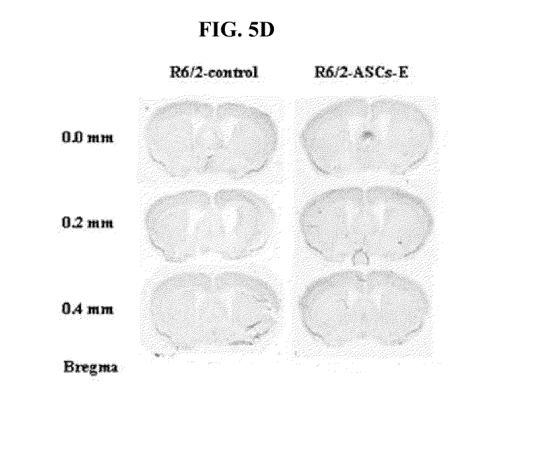

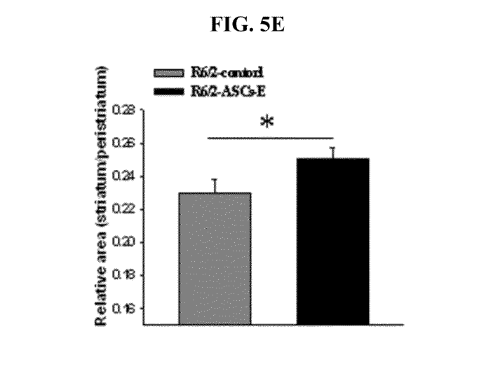

R6/2 mouse shows striatal atrophy and mHtt aggregation in striatum and cortex during disease progression. To examine histological changes of the brain, volume of striatum and mHtt aggregation were evaluated at 12 weeks of age. As described in the materials and method section, the effect on mHtt aggregation, brains were sectioned and sliced tissues were stained with an EM48 antibody which detects aggregation of mutant huntingtin. As shown in FIG. 5A, injection of ASCs-E reduced mHtt aggregation in the striatum but not in the cortex. The protein was extracted from R6/2 mouse brain and mHtt aggregation was measured by western blot. ASCs-E treated group showed decreased mHtt aggregation in the brain compared with vehicle treated group (FIG. 5B, C). Volumes of striatum and peristriatum were measured respectively and the ratio of striatum to peristriatum was calculated. We found that ASCs-E injected R6/2 brain showed an increased ratio of striatum to peristriatum compared with vehicle injected R6/2 brain (0.22±0.01 vs. 0.25±0.01, p<0.01, n=7) (FIGS. 5D and E).

These results indicate that the present hASCs-E suppress the progression of the disease by regulating the pathological mechanism of the Huntington's disease.

### Example 6

### Activation of CREB and PGC-1a in R6/2 Mice and Neuronal Cells by hASCs-E

Dysfunction of pCREB (cAMP-responsive element binding protein)-PGC-1α (peroxisome proliferator-activated receptor-γ coactivator 1) pathway has been regarded as the key molecules for HD progression (McGill J K, Beal M F. 2006. Cell 127: 465-468; Okamoto S, et al. 2009. Nat Med 15: 1407-1413). The suppression of the CREB activity leads to the PGC-1 malfunction which in turn results in striatal atrophy in HD by the glutamate toxicity (Cui L, et al. 2006. Cell 127:59-69). Considering that the activation of PGC-1 α provides a protective effect from the damages caused by cell toxicity and mitochondrial malfunction induced by mHtt, the activation of pCREB)-PGC-1α pathway can slow down the development of HD and/or the progression of HD.

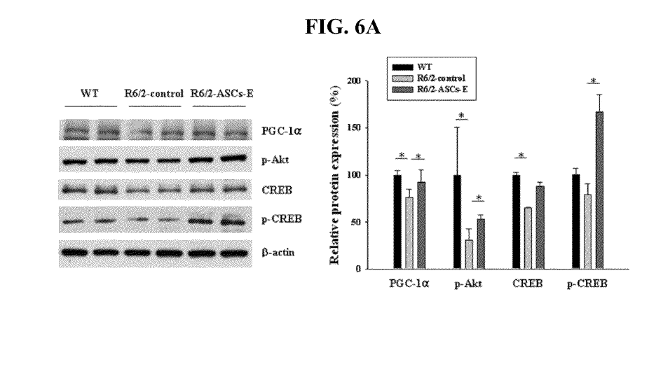

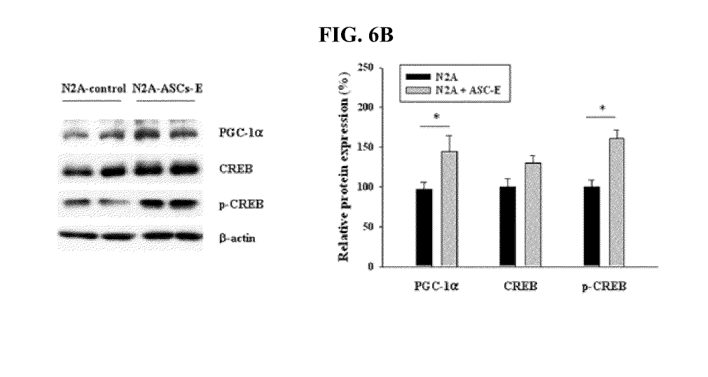

Therefore, to examine effects of ASCs-E injection on this pathway, 12 weeks old R6/2 mice treated with control vehicle or ASCs-E for 6 weeks were sacrificed and western blot analysis was performed. Transplantation of ASCs-E promoted expression of p-Akt, p-CREB and PGC-1a (p, 0.05 vs. R6/2 control), whereas they were decreased in R6/2 control mice (FIG. 6A). To examine in vitro effects of ASCs-E, neuro2A cells were cultured and incubated with ASCs-E for 1 day.

Then cells were harvested and levels of p-Akt, p-CREB and PGC1a were measured by western blot. In ASCs-E treated cells, levels of p-CREB and PGC1a were significantly increased (FIG. 6B). Overall, neuroprotective p-CREB-PGC1 α pathway was activated by treatment of ASCs-E. These results indicate that the hASCs-E has a neuroprotective and thus therapeutic activity through the activation of p-CREB-PGC1 α pathway.

### Example 7

### Altered Gene Expression by hASC-E Treatment

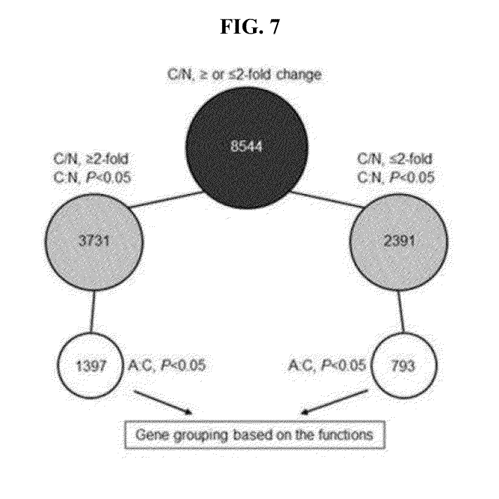

To examine cellular and molecular changes, a gene expression microarray experiment was performed with the brains of rats subjected to permanent focal cerebral ischemia, in which hASCs-E was treated at 1 h after the insult. Results are shown in FIG. 7A, and Tables 1 and 2, in which it was found that the expression of many genes related to inflammation or immune responses was substantially downregulated in the ischemic rats treated with hASCs-E, compared to those in the control (Table 1). In addition, the expression of genes related to cell-death or apoptosis was prevented or inhibited in ischemic brains treated with the hASCs-E (Table 2).

In table 1, the genes related to inflammatory and immune responses, which show a statistical difference (P<0.05, Mann-Whitney U test) in the level of the expression and (N, normal; C, control; A, hASCs-E). The values indicate fold differences expressed in the ratio of C:N (C/N) or A:C (A/C), respectively.

TABLE 1

| Name | C/N | A/N | A/C |
|---|---|---|---|
| “Nitric oxide synthase 2, inducible” | 2.760407816 | 14.97026338 | 5.423207141 |
| Kininogen 1 | 350.1478415 | 674.512123 | 1.92636379 |
| “Fibrinogen, gamma polypeptide” | 3.850746269 | 7.084577114 | 1.839793282 |
| “Integrin, beta 6” | 0.4280092 | 0.77991311 | 1.822187724 |
| Chemokine (C-C motif) receptor 1 | 15.06466877 | 27.21608833 | 1.806617108 |
| “Fibrinogen, gamma polypeptide” | 3.162878788 | 5.507575758 | 1.741317365 |
| Cold autoinflammatory syndrome 1 homolog (human) | 2.056179775 | 3.481273408 | 1.693078324 |
| (predicted) | | | |
| “Myosin, heavy polypeptide 9, non-muscle” | 2.393056417 | 3.596404216 | 1.502849741 |
| Annexin A1 | 2.966007343 | 4.454696198 | 1.50191678 |
| Kininogen 1 | 552.3961661 | 822.0447284 | 1.488143436 |
| Chemokine (C-X-C motif) ligand 10 | 2.776985413 | 3.906482982 | 1.406735146 |
| Annexin A1 | 3.234025974 | 4.473506494 | 1.383262389 |
| “Tumor necrosis factor (TNF superfamily, member 2)” | 3.466738197 | 4.442060086 | 1.281337047 |
| “Rattus norvegicus tumor necrosis factor receptor | 3.970288115 | 5.049519808 | 1.271827047 |
| superfamily, member 1b (Tnfrsf1b), mRNA | | | |
| [NM_130426]” | | | |
| “Tumor necrosis factor receptor superfamily, member | 4.44399338 | 3.234550167 | 0.727847657 |
| 1a” | | | |
| Chemokine (C-C motif) ligand 2 | 300.3833516 | 213.143483 | 0.709571559 |
| Mitogen activated protein kinase kinase 3 | 2.774409545 | 1.896761626 | 0.683663171 |
| “Nuclear factor of activated T-cells, cytoplasmic, | 2.637477585 | 1.783323371 | 0.676147309 |
| calcineurin-dependent 4” | | | |
| Spleen tyrosine kinase | 2.568902756 | 1.65574623 | 0.644534413 |
| “PE23_RAT (P34980) Prostaglandin E2 receptor, EP3 | 4.602122424 | 2.911411873 | 0.632623734 |
| subtype (Prostanoid EP3 receptor) (PGE receptor, EP3 | | | |
| subtype), partial (8%) [TC559337]” | | | |
| Zinc finger protein 36 | 23.44980588 | 14.81253466 | 0.63166982 |
| Toll-like receptor 5 | 2.24691358 | 1.416904084 | 0.630600169 |
| “PREDICTED: Rattus norvegicus matrix | 2.690807799 | 1.674651811 | 0.622360248 |
| metalloproteinase 25 (predicted) (Mmp25_predicted), | | | |
| mRNA [XM_220199]” | | | |
| Chemokine (C-C motif) ligand 24 | 3.668046929 | 2.259489303 | 0.615992474 |
| “Transglutaminase 2, C polypeptide” | 6.273610886 | 3.701117771 | 0.589950164 |
| “Transforming growth factor, beta 1” | 5.581350834 | 3.13234892 | 0.561216991 |
| Adenosine A2a receptor | 0.275431332 | 0.143909593 | 0.522488098 |
| Surfactant associated protein D | 2.267884323 | 1.070522577 | 0.472035794 |
| “Selectin, endothelial cell” | 36.52444871 | 17.08373282 | 0.467734173 |
| Adenosine A2a receptor | 0.273663489 | 0.124844592 | 0.456197471 |
| Chemokine (C-C motif) ligand 22 | 4.135620915 | 1.799019608 | 0.435005927 |
| Interleukin 4 | 5.766509434 | 1.925707547 | 0.33394683 |
| “Tumor necrosis factor receptor superfamily, member | 2.443911793 | 0.741131352 | 0.303256179 |
| 4” | | | |
| Chemokine (C-C motif) ligand 22 | 5.638115632 | 1.643825839 | 0.291555893 |
| “Serine (or cysteine) peptidase inhibitor, clade C | 2.11882716 | 0.462962963 | 0.218499636 |
| (antithrombin), member 1” | | | |
| Endothelial-specific receptor tyrosine kinase | 4.625761532 | 0.43202785 | 0.093396049 |
| “Nitric oxide synthase 2, inducible” | 2.760407816 | 14.97026338 | 5.423207141 |
| CD4 antigen | 0.270816158 | 1.206512778 | 4.455098935 |
| Similar to Map4k6-pending protein | 0.251638931 | 0.765002521 | 3.04008016 |
| “Chitinase, acidic” | 0.19297597 | 0.56023467 | 2.903131767 |
| “PREDICTED: Rattus norvegicus similar to | 2.971119134 | 8.113718412 | 2.730862697 |
| Hypothetical protein E430029F06 (LOC305139), | | | |
| mRNA [XM_223172]” | | | |
| “Tumor necrosis factor receptor superfamily, member 6” | 3.025261861 | 8.157732594 | 2.696537678 |
| Complement component 6 | 3.905747126 | 10.03218391 | 2.568569747 |
| Colony stimulating factor 3 (granulocyte) | 73.9 | 183.7666667 | 2.48669373 |
| IgA Fc receptor | 6.74186551 | 15.82646421 | 2.347490347 |
| Collectin sub-family member 12 | 0.444257045 | 0.910333049 | 2.049113364 |
| Similar to alpha-1 major acute phase protein | 339.8471616 | 688.2641921 | 2.025216833 |
| prepeptide | | | |
| Butyrophilin-like 8 | 2.263333333 | 4.56 | 2.014727541 |
| “Rattus norvegicus schlafen 3 (Slfn3), mRNA | 9.863049096 | 19.21317829 | 1.947995808 |
| [NM_053687]” | | | |
| Kininogen 1 | 350.1478415 | 674.512123 | 1.92636379 |
| CD69 antigen | 3.396825397 | 6.455026455 | 1.900311526 |
| “Fibrinogen, gamma polypeptide” | 3.850746269 | 7.084577114 | 1.839793282 |
| “Integrin, beta 6” | 0.4280092 | 0.77991311 | 1.822187724 |
| Chemokine (C-C motif) receptor 1 | 15.06466877 | 27.21608833 | 1.806617108 |
| “Fc fragment of IgG, low affinity IIIa, receptor” | 2.834568201 | 5.032187538 | 1.775292454 |
| “Fibrinogen, gamma polypeptide” | 3.162878788 | 5.507575758 | 1.741317365 |
| Cold autoinflammatory syndrome 1 homolog (human) | 2.056179775 | 3.481273408 | 1.693078324 |
| (predicted) | | | |
| Pancreatic lipase-related protein 2 | 0.373477407 | 0.611591356 | 1.637559179 |
| Chemokine (C-X-C motif) ligand 11 | 3.500687758 | 5.640990371 | 1.611394892 |
| Interleukin 18 | 2.48544131 | 3.967470428 | 1.596284093 |
| Parathymosin | 0.49141549 | 0.765356734 | 1.557453416 |
| Phospholipid scramblase 1 | 3.234767025 | 4.951812027 | 1.53080948 |
| “RT1 class II, locus Da” | 2.182625 | 3.316625 | 1.519557872 |
| “Myosin, heavy polypeptide 9, non-muscle” | 2.393056417 | 3.596404216 | 1.502849741 |
| Annexin A1 | 2.966007343 | 4.454696198 | 1.50191678 |
| Kininogen 1 | 552.3961661 | 822.0447284 | 1.488143436 |
| Lymphocyte cytosolic protein 2 | 4.080607477 | 5.992211838 | 1.468460731 |
| “Opioid receptor, kappa 1” | 0.361549708 | 0.52997076 | 1.465830975 |
| Interleukin 6 | 111.4482759 | 163.256705 | 1.464865237 |
| “Tumor necrosis factor receptor superfamily, member 6” | 3.166712329 | 4.526986301 | 1.429554008 |
| Chemokine (C-X-C motif) ligand 10 | 2.776985413 | 3.906482982 | 1.406735146 |
| Cathepsin C | 3.70887559 | 5.161014534 | 1.391530778 |
| Annexin A1 | 3.234025974 | 4.473506494 | 1.383262389 |
| “Serine (or cysteine) peptidase inhibitor, clade G, | 2.380028984 | 3.288937961 | 1.381889878 |
| member 1” | | | |
| “RT1 class II, locus Da” | 2.354012043 | 3.19889838 | 1.358913345 |
| “Tumor necrosis factor (TNF superfamily, member 2)” | 3.466738197 | 4.442060086 | 1.281337047 |
| “Rattus norvegicus tumor necrosis factor receptor | 3.970288115 | 5.049519808 | 1.271827047 |
| superfamily, member 1b (Tnfrsf1b), mRNA | | | |
| [NM_130426]” | | | |
| Neutrophil cytosolic factor 2 (predicted) | 10.86848635 | 13.42431762 | 1.235159817 |
| Cystatin F (leukocystatin) (predicted) | 19.13937282 | 17.04878049 | 0.890770071 |
| “RT1 class II, locus Bb” | 3.321760621 | 2.663292927 | 0.801771479 |
| “Interleukin 6 receptor, alpha” | 2.095234107 | 1.645028897 | 0.785128923 |
| “Superoxide dismutase 2, mitochondrial” | 2.024089207 | 1.565202588 | 0.773287354 |
| Fos-like antigen 1 | 163.2388664 | 125.8704453 | 0.771081349 |
| Similar to hypothetical protein FLJ14466 | 2.153382233 | 1.602444988 | 0.7441526 |
| “Mannose binding lectin 1, protein A” | 2.562982005 | 1.87403599 | 0.731193581 |
| “Tumor necrosis factor receptor superfamily, member 1a” | 4.44399338 | 3.234550167 | 0.727847657 |
| Chemokine (C-C motif) ligand 2 | 300.3833516 | 213.143483 | 0.709571559 |
| Signal transducer and activator of transcription 3 | 3.567073171 | 2.515142276 | 0.705099715 |
| Vascular endothelial growth factor A | 4.515312916 | 3.13870395 | 0.695124349 |
| Coagulation factor II | 7.217391304 | 5.016830295 | 0.695102993 |
| Mitogen activated protein kinase kinase 3 | 2.774409545 | 1.896761626 | 0.683663171 |
| “Nuclear factor of activated T-cells, cytoplasmic, | 2.637477585 | 1.783323371 | 0.676147309 |
| calcineurin-dependent 4” | | | |
| “Nuclear factor, interleukin 3 regulated” | 5.311470715 | 3.510668767 | 0.660959827 |
| Cyclin-dependent kinase inhibitor 1A | 6.98879203 | 4.616438356 | 0.660548824 |
| “PREDICTED: Rattus norvegicus mannan-binding | 3.871146904 | 2.543737851 | 0.657101865 |
| lectin serine protease 2 (Masp2), mRNA | | | |
| [XM_342977]” | | | |
| Spleen tyrosine kinase | 2.568902756 | 1.65574623 | 0.644534413 |
| “PREDICTED: Rattus norvegicus growth arrest and | 9.211682038 | 5.8304787 | 0.632943981 |
| DNA-damage-inducible 45 gamma (predicted) | | | |
| (Gadd45g_predicted), mRNA [XM_237999]” | | | |
| “PE23_RAT (P34980) Prostaglandin E2 receptor, EP3 | 4.602122424 | 2.911411873 | 0.632623734 |
| subtype (Prostanoid EP3 receptor) (PGE receptor, EP3 | | | |
| subtype), partial (8%) [TC559337]” | | | |
| Zinc finger protein 36 | 23.44980588 | 14.81253466 | 0.63166982 |
| Toll-like receptor 5 | 2.24691358 | 1.416904084 | 0.630600169 |
| Immediate early response 3 | 4.680180387 | 2.947862501 | 0.629860872 |
| “PREDICTED: Rattus norvegicus matrix | 2.690807799 | 1.674651811 | 0.622360248 |
| metalloproteinase 25 (predicted) (Mmp25_predicted), | | | |
| mRNA [XM_220199]” | | | |
| SH2 domain protein 2A | 6.337868481 | 3.926303855 | 0.619499106 |
| Single immunoglobulin and toll-interleukin 1 receptor | 4.476715485 | 2.772256047 | 0.619261165 |
| (TIR) domain | | | |
| Chemokine (C-C motif) ligand 24 | 3.668046929 | 2.259489303 | 0.615992474 |
| Transferrin | 2.203634715 | 1.355298384 | 0.615028604 |
| “Transglutaminase 2, C polypeptide” | 6.273610886 | 3.701117771 | 0.589950164 |
| Zinc finger protein 179 | 0.45854035 | 0.266123197 | 0.580370292 |
| “Q7M5K8 (Q7M5K8) E3 CR1-gamma1, partial (6%) | 6.083615238 | 3.509339609 | 0.576851012 |
| [TC554433]” | | | |
| “Transforming growth factor, beta 1” | 5.581350834 | 3.13234892 | 0.561216991 |
| CD7 antigen (predicted) | 3.04180602 | 1.690635452 | 0.55579989 |
| “Histocompatibility 2, T region locus 18” | 2.244856999 | 1.182137481 | 0.526598122 |
| Adenosine A2a receptor | 0.275431332 | 0.143909593 | 0.522488098 |
| CD7 antigen (predicted) | 3.525684932 | 1.815068493 | 0.514813016 |
| Csk binding protein | 2.025697102 | 0.96172772 | 0.474763833 |
| Zeta-chain (TCR) associated protein kinase 70 | 2.557060616 | 1.213003725 | 0.474374255 |
| (mapped) | | | |
| Surfactant associated protein D | 2.267884323 | 1.070522577 | 0.472035794 |
| “Selectin, endothelial cell” | 36.52444871 | 17.08373282 | 0.467734173 |
| Similar to KIAA1086 protein (predicted) | 0.489209074 | 0.22624157 | 0.462463968 |
| Complexin 2 | 0.364334086 | 0.166591422 | 0.457249071 |
| Adenosine A2a receptor | 0.273663489 | 0.124844592 | 0.456197471 |
| Hepcidin antimicrobial peptide | 3.291360294 | 1.481617647 | 0.450153588 |
| Programmed cell death 1 ligand 2 (predicted) | 3.040540541 | 1.345720721 | 0.442592593 |
| Chemokine (C-C motif) ligand 22 | 4.135620915 | 1.799019608 | 0.435005927 |
| “RT1 class I, M10, gene 1” | 3.032581454 | 1.274853801 | 0.420385675 |
| “PREDICTED: Rattus norvegicus similar to IL-27 | 2.173186813 | 0.905758242 | 0.416788026 |
| p28 subunit (LOC365368), mRNA [XM_344962]” | | | |
| Chemokine (C-C motif) ligand 17 | 3.627866896 | 1.491183879 | 0.411035995 |
| “RT1 class Ib, locus Aw2” | 3.728688525 | 1.472131148 | 0.394812047 |
| Non-catalytic region of tyrosine kinase adaptor protein | 3.225402504 | 1.237924866 | 0.38380477 |
| 2 (predicted) | | | |
| “RT1 class II, locus Ba” | 3.882047959 | 1.406351264 | 0.362270451 |
| Inhibin beta-A | 20.54924681 | 7.194901506 | 0.350129694 |
| SFFV proviral integration 1 | 5.139111709 | 1.756393001 | 0.343305035 |
| Interleukin 4 | 5.766509434 | 1.925707547 | 0.33394683 |
| “Tumor necrosis factor receptor superfamily, member 4” | 2.443911793 | 0.741131352 | 0.303256179 |
| Complement component 9 | 2.189082724 | 0.662352279 | 0.302570694 |
| Chemokine (C-C motif) ligand 22 | 5.638115632 | 1.643825839 | 0.291555893 |
| E-3 epididymal fluid protein | 2.279507603 | 0.517740768 | 0.227128335 |
| “Serine (or cysteine) peptidase inhibitor, clade C | 2.11882716 | 0.462962963 | 0.218499636 |
| (antithrombin), member 1” | | | |
| Cannabinoid receptor 2 (macrophage) | 3.879177378 | 0.547943445 | 0.141252485 |
| “NK2 transcription factor related, locus 3 (Drosophila) | 5.172535211 | 0.698943662 | 0.135125936 |
| (predicted)” | | | |
| Interleukin 3 | 9.042750929 | 1.090613383 | 0.120606372 |
| Endothelial-specific receptor tyrosine kinase | 4.625761532 | 0.43202785 | 0.093396049 |

In table 2, the genes related to cell-death or apoptosis processes, which show a statistical difference (P<0.05, Mann-Whitney U test) in the level of the expression and (N, normal; C; A, hASCs-E). The values indicate fold differences expressed in the ratio of C:N (C/N) or A:C (A/C), respectively.

TABLE 2

| Name | C/N | A/N | A/C |
|---|---|---|---|
| “GULP, engulfment adaptor PTB domain | 0.355381166 | 1.899303139 | 5.34384858 |
| containing 1” | | | |
| Snail homolog 2 (Drosophila) | 0.211498973 | 1.068788501 | 5.053398058 |
| Rac/cdc42 guanine nucleotide exchange factor 6 | 0.265817748 | 0.985674493 | 3.708083832 |
| “Tumor necrosis factor receptor superfamily, | 3.025261861 | 8.157732594 | 2.696537678 |
| member 6” | | | |
| Complement component 6 | 3.905747126 | 10.03218391 | 2.568569747 |
| “Engulfment and cell motility 1, ced-12 homolog | 0.316743069 | 0.710325256 | 2.242591316 |
| (C. elegans) (predicted)” | | | |
| Parathyroid hormone | 0.247176509 | 0.502420136 | 2.032637076 |
| “Transcription factor AP-2, alpha (predicted)” | 3.334987593 | 6.322580645 | 1.895833333 |
| Caspase 1 | 2.056037441 | 3.885803432 | 1.889947797 |
| Cold autoinflammatory syndrome 1 homolog | 2.056179775 | 3.481273408 | 1.693078324 |
| (human) (predicted) | | | |
| Axin2 | 0.398222222 | 0.659555556 | 1.65625 |
| Gonadotropin-releasing hormone 1 | 0.254872881 | 0.419279661 | 1.645054032 |
| Apoptosis inhibitor 5 (predicted) | 0.394309025 | 0.644439968 | 1.634352569 |
| CAMP responsive element binding protein 1 | 2.220659816 | 3.59951325 | 1.620920604 |
| Interleukin 18 | 2.48544131 | 3.967470428 | 1.596284093 |
| CAMP responsive element binding protein 1 | 2.127654498 | 3.390598902 | 1.593585287 |
| Gap junction membrane channel protein beta 6 | 0.337004099 | 0.516431599 | 1.532419338 |
| Annexin A1 | 2.966007343 | 4.454696198 | 1.50191678 |
| Interleukin 6 | 111.4482759 | 163.256705 | 1.464865237 |
| Lysozyme | 3.075546275 | 4.408233276 | 1.433317168 |
| “Tumor necrosis factor receptor superfamily, | 3.166712329 | 4.526986301 | 1.429554008 |
| member 6” | | | |
| Annexin A1 | 3.234025974 | 4.473506494 | 1.383262389 |
| Glucagon-like peptide 1 receptor | 0.476292444 | 0.648210462 | 1.360950546 |
| Myelocytomatosis viral oncogene homolog | 7.10543213 | 9.393861564 | 1.322067594 |
| (avian) | | | |
| “Tumor necrosis factor (TNF superfamily, | 3.466738197 | 4.442060086 | 1.281337047 |
| member 2)” | | | |
| Fibroblast growth factor 2 | 2.378138848 | 3.037912358 | 1.277432712 |
| “Rattus norvegicus tumor necrosis factor receptor | 3.970288115 | 5.049519808 | 1.271827047 |
| superfamily, member 1b (Tnfrsf1b), mRNA | | | |
| [NM_130426]” | | | |
| “Nuclear factor of kappa light chain gene | 3.559615385 | 2.830288462 | 0.795110751 |
| enhancer in B-cells inhibitor, alpha” | | | |
| “Rattus norvegicus eukaryotic translation | 2.296498869 | 1.810324454 | 0.78829756 |
| initiation factor 4 gamma, 2 (Eif4g2), mRNA | | | |
| [NM_001017374]” | | | |
| Heat shock 70 kD protein 1A | 43.87099449 | 34.41114289 | 0.784371161 |
| “Superoxide dismutase 2, mitochondrial” | 2.024089207 | 1.565202588 | 0.773287354 |
| “Actinin, alpha 1” | 2.282334734 | 1.760778245 | 0.771481159 |
| “Sodium channel, voltage-gated, type 2, alpha 1 | 0.503154789 | 0.377462551 | 0.750191709 |
| polypeptide” | | | |
| “Tumor necrosis factor receptor superfamily, | 4.44399338 | 3.234550167 | 0.727847657 |
| member 1a” | | | |
| Chemokine (C-C motif) ligand 2 | 300.3833516 | 213.143483 | 0.709571559 |
| Similar to livin inhibitor of apoptosis isoform | 0.366433022 | 0.257788162 | 0.703506908 |
| beta (predicted) | | | |
| Angiopoietin-like 4 | 6.498098859 | 4.571187157 | 0.703465314 |
| Vascular endothelial growth factor A | 4.515312916 | 3.13870395 | 0.695124349 |
| Endothelin converting enzyme 1 | 2.575155494 | 1.728921907 | 0.671385441 |
| Cyclin-dependent kinase inhibitor 1A | 6.98879203 | 4.616438356 | 0.660548824 |
| “Actinin, alpha 1” | 2.281856252 | 1.498248013 | 0.65659176 |
| “Tumor necrosis factor receptor superfamily, | 21.42399267 | 13.6790293 | 0.638491131 |
| member 12a” | | | |
| “PREDICTED: Rattus norvegicus growth arrest | 9.211682038 | 5.8304787 | 0.632943981 |
| and DNA-damage-inducible 45 gamma | | | |
| (predicted) (Gadd45g_predicted), mRNA | | | |
| [XM_237999]” | | | |
| “Transglutaminase 2, C polypeptide” | 6.273610886 | 3.701117771 | 0.589950164 |
| “BH3 interacting (with BCL2 family) domain, | 0.491590466 | 0.289498498 | 0.588901775 |
| apoptosis agonist” | | | |
| “Q7M5K8 (Q7M5K8) E3 CR1-gamma1, partial | 6.083615238 | 3.509339609 | 0.576851012 |
| (6%) [TC554433]” | | | |
| “Tumor necrosis factor receptor superfamily, | 21.36997258 | 12.09209324 | 0.565845052 |
| member 12a” | | | |
| “Transforming growth factor, beta 1” | 5.581350834 | 3.13234892 | 0.561216991 |
| Growth arrest and DNA-damage-inducible 45 | 8.401654757 | 4.685031967 | 0.55763205 |
| beta | | | |
| “Phosphodiesterase 1B, Ca2 + calmodulin | 0.470164492 | 0.260748724 | 0.554590421 |
| dependent” | | | |
| “Lectin, galactose binding, soluble 7” | 2.735493988 | 1.490067956 | 0.544716224 |
| “D4, zinc and double PHD fingers family 2 | 2.050434783 | 1.114534161 | 0.543559918 |
| (predicted)” | | | |
| “Procollagen, type XVIII, alpha I” | 2.746459818 | 1.483900063 | 0.540295567 |
| “Nerve growth factor receptor (TNFR | 2.383068378 | 1.280597587 | 0.537373413 |
| superfamily, member 16)” | | | |
| DNA-damage inducible transcript 3 | 2.652206526 | 1.422302788 | 0.536271506 |
| “Procollagen, type XVIII, alpha 1” | 2.794282632 | 1.491585761 | 0.533799174 |
| Adenosine A2a receptor | 0.275431332 | 0.143909593 | 0.522488098 |
| Granzyme B | 2.95033358 | 1.527057079 | 0.51758794 |
| Fibroblast growth factor 4 | 2.104719764 | 1.08480826 | 0.515416959 |
| “PREDICTED: Rattus norvegicus similar to | 2.540805223 | 1.218715996 | 0.479657388 |
| Natural killer cell protease 1 precursor (RNKP-1) | | | |
| (Granzyme B) (LOC290262), mRNA | | | |
| [XM_224224]” | | | |
| Placentae and embryos oncofetal gene | 2.918565558 | 1.343668285 | 0.460386535 |
| Adenosine A2a receptor | 0.273663489 | 0.124844592 | 0.456197471 |
| LPS-induced TN factor | 2.10547504 | 0.807971014 | 0.38374761 |
| Cell division cycle 2 homolog (S. pombe)-like 1 | 2.694433732 | 0.988173287 | 0.366746183 |
| Inhibin beta-A | 20.54924681 | 7.194901506 | 0.350129694 |
| Interleukin 4 | 5.766509434 | 1.925707547 | 0.33394683 |
| “Myc-like oncogene, s-myc protein” | 2.229032258 | 0.700967742 | 0.31447178 |
| “Tumor necrosis factor receptor superfamily, | 2.443911793 | 0.741131352 | 0.303256179 |
| member 4” | | | |
| Complement component 9 | 2.189082724 | 0.662352279 | 0.302570694 |
| Granzyme B | 3.415121255 | 0.864479315 | 0.253132832 |
| Interleukin 3 | 9.042750929 | 1.090613383 | 0.120606372 |
| “GULP, engulfment adaptor PTB domain | 0.355381166 | 1.899103139 | 5.34384858 |
| containing 1” | | | |
| Snail homolog 2 (Drosophila) | 0.211498973 | 1.068788501 | 5.053398058 |
| Rac/cdc42 guanine nucleotide exchange factor 6 | 0.265817748 | 0.985674493 | 3.708083832 |
| “Tumor necrosis factor receptor superfamily, | 3.025261861 | 8.157732594 | 2.696537678 |
| member 6” | | | |
| Complement component 6 | 3.905747126 | 10.03218391 | 2.568569747 |
| “Engulfment and cell motility 1, ced-12 homolog | 0.316743069 | 0.710325256 | 2.242591316 |
| (C. elegans) (predicted)” | | | |
| Parathyroid hormone | 0.247176509 | 0.502420136 | 2.032637076 |
| “Transcription factor AP-2, alpha (predicted)” | 3.334987593 | 6.322580645 | 1.895833333 |
| Caspase 1 | 2.056037441 | 3.885803432 | 1.889947797 |
| Cold autoinflammatory syndrome 1 homolog | 2.056179775 | 3.481273408 | 1.693078324 |
| (human) (predicted) | | | |
| Gonadotropin-releasing hormone 1 | 0.254872881 | 0.419279661 | 1.645054032 |
| Apoptosis inhibitor 5 (predicted) | 0.394309025 | 0.644439968 | 1.634352569 |
| CAMP responsive element binding protein 1 | 2.220659816 | 3.59951325 | 1.620920604 |
| Interleukin 18 | 2.48544131 | 3.967470428 | 1.596284093 |
| CAMP responsive element binding protein 1 | 2.127654498 | 3.390598902 | 1.593585287 |
| Gap junction membrane channel protein beta 6 | 0.337004099 | 0.516431599 | 1.532419338 |
| Annexin A1 | 2.966007343 | 4.454696198 | 1.50191678 |
| Interleukin 6 | 111.4482759 | 163.256705 | 1.464865237 |
| “Tumor necrosis factor receptor superfamily, | 3.166712329 | 4.526986301 | 1.429554008 |
| member 6” | | | |
| Annexin A1 | 3.234025974 | 4.473506494 | 1.383262389 |
| Glucagon-like peptide 1 receptor | 0.476292444 | 0.648210462 | 1.360950546 |
| Myelocytomatosis viral oncogene homolog | 7.10543213 | 9.393861564 | 1.322067594 |
| (avian) | | | |
| “Tumor necrosis factor (TNF superfamily, | 3.466738197 | 4.442060086 | 1.281337047 |
| member 2)” | | | |
| “Nuclear factor of kappa light chain gene | 3.559615385 | 2.830288462 | 0.795110751 |
| enhancer in B-cells inhibitor, alpha” | | | |
| Heat shock 70 kD protein 1A | 43.87099449 | 34.41114289 | 0.784371161 |
| “Superoxide dismutase 2, mitochondrial” | 2.024089207 | 1.565202588 | 0.773287354 |
| “Actinin, alpha 1” | 2.282334734 | 1.760778245 | 0.771481159 |
| “Sodium channel, voltage-gated, type 2, alpha 1 | 0.503154789 | 0.377462551 | 0.750191709 |
| polypeptide” | | | |
| “Tumor necrosis factor receptor superfamily, | 4.44399338 | 3.234550167 | 0.727847657 |
| member 1a” | | | |
| Chemokine (C-C motif) ligand 2 | 300.3833516 | 213.143483 | 0.709571559 |
| Similar to livin inhibitor of apoptosis isoform | 0.366433022 | 0.257788162 | 0.703506908 |
| beta (predicted) | | | |
| Angiopoietin-like 4 | 6.498098859 | 4.571187157 | 0.703465314 |
| Vascular endothelial growth factor A | 4.515312916 | 3.13870395 | 0.695124349 |
| Endothelin converting enzyme 1 | 2.575155494 | 1.728921907 | 0.671385441 |
| Cyclin-dependent kinase inhibitor 1A | 6.98879203 | 4.616438356 | 0.660548824 |
| “Actinin, alpha 1” | 2.281856252 | 1.498248013 | 0.65659176 |
| “PREDICTED: Rattus norvegicus growth arrest | 9.211682038 | 5.8304787 | 0.632943981 |
| and DNA-damage-inducible 45 gamma | | | |
| (predicted) (Gadd45g_predicted), mRNA | | | |
| [XM_237999]” | | | |
| “Transglutaminase 2, C polypeptide” | 6.273610886 | 3.701117771 | 0.589950164 |
| “BH3 interacting (with BCL2 family) domain, | 0.491590466 | 0.289498498 | 0.588903775 |
| apoptosis agonist” | | | |
| “Q7M5K8 (Q7M5K8) E3 CR1-gamma1, partial | 6.083615238 | 3.509339609 | 0.576851012 |
| (6%) [TC554433J” | | | |
| “Transforming growth factor, beta 1” | 5.581350834 | 3.13234892 | 0.561216991 |
| Growth arrest and DNA-damage-inducible 45 | 8.401654757 | 4.685031967 | 0.55763205 |
| beta | | | |
| “Phosphodiesterase 1B, Ca2 + calmodulin | 0.470164492 | 0.260748724 | 0.554590421 |
| dependent” | | | |
| “Lectin, galactose binding, soluble 7” | 2.735493988 | 1.490067956 | 0.544716224 |
| “D4, zinc and double PHD fingers family 2 | 2.050434783 | 1.114534161 | 0.543559918 |
| (predicted)” | | | |
| “Procollagen, type XVIII, alpha 1” | 2.746459818 | 1.483900063 | 0.540295567 |
| “Nerve growth factor receptor (TNFR | 2.383068378 | 1.280597587 | 0.537373413 |
| superfamily, member 16)” | | | |
| DNA-damage inducible transcript 3 | 2.652206526 | 1.422302788 | 0.536271506 |
| “Procollagen, type XVIII, alpha 1” | 2.794282632 | 1.491585761 | 0.533799174 |
| Adenosine A2a receptor | 0.275431332 | 0.143909593 | 0.522488098 |
| Granzyme B | 2.95033358 | 1.527057079 | 0.51758794 |
| Fibroblast growth factor 4 | 2.104719764 | 1.08480826 | 0.515416959 |
| — | 8.232451679 | 4.174465921 | 0.507074452 |
| “PREDICTED: Rattus norvegicus similar to | 2.540805223 | 1.218715996 | 0.479657388 |
| Natural killer cell protease 1 precursor (RNKP-1) | | | |
| (Granzyme B) (LOC290262), mRNA | | | |
| [XM_224224]” | | | |
| Adenosine A2a receptor | 0.273663489 | 0.124844592 | 0.456197471 |
| LPS-induced TN factor | 2.10547504 | 0.807971014 | 0.38374761 |
| Cell division cycle 2 homolog (S. pombe)-like 1 | 2.694433732 | 0.988173287 | 0.366746183 |
| Inhibin beta-A | 20.54924681 | 7.194901506 | 0.350129694 |
| Interleukin 4 | 5.766509434 | 1.925707547 | 0.33394683 |
| “Myc-like oncogene, s-myc protein” | 2.229032258 | 0.700967742 | 0.31447178 |
| “Tumor necrosis factor receptor superfamily, | 2.443911793 | 0.741131352 | 0.303256179 |
| member 4” | | | |
| Complement component 9 | 2.189082724 | 0.662352279 | 0.302570694 |
| Granzyme B | 3.415121255 | 0.864479315 | 0.253132832 |
| Interleukin 3 | 9.042750929 | 1.090613383 | 0.120606372 |

The same results as above were obtained using in vitro Neuro2a cells in gene expression microarray after concurrent treatments of hASC-E with OGD. In comparisons between OGD+hASC-E and OGD+control, the genes related to inflammatory or immune responses constituted the largest portion among the genes changed more than 2 fold by hASC-E treatment (Data not shown).

In summary, as shown in Table 1 and 2, the expression of many genes related to inflammation, immune response, and cell-death was changed substantially in the ischemic rats or neuronal cells treated with the hASCs-E.

Results as described herein reveal a neuroprotective role of hASCs-E in diseases such as stroke, HD and thus ASCs-E can be used advantageously for a stem cell-based, noninvasive therapy for treating neurologic disease such as stroke and HD. Also, in contrast to the stem cell transplantation or therapies, the present method or composition enable the repeated treatments, due to the convenient administration, thus leading to a more efficient prevention and/or curing of the disease of interest.

The various singular/plural permutations may be expressly set forth herein for sake of clarity. Although a few embodiments of the present disclosure have been shown and described, it would be appreciated by those skilled in the art that changes may be made in this embodiment without departing from the principles and sprit of the invention, the scope of which is defined in the claims and their equivalents.

What is claimed is:

1. A method of treating or preventing neurologic disease in a subject in need thereof comprising administering an effective amount of adipose stem cell extract to the subject.

2. The method of claim 1, wherein the adipose stem cell extract activates pCREB-PGC-1alpha pathway.

3. The method of claim 1, wherein the adipose stem cell extract regulates inflammatory response or suppresses neuronal cell-death.

4. The method of claim 1, wherein the stem cell is autologous adipose stem cell, heterologous adipose stem cell, or allogenic adipose stem cell.

5. The method of claim 1 wherein the extract comprises a soluble and/or an insoluble fraction, wherein the soluble fraction comprises a protein, polypeptide or peptide.

6. The method of claim 1, wherein the therapeutic effect of the extract is exhibited by a protein, polypeptide or peptide comprised in the extract.

7. The method of claim 1, wherein the neurologic disease comprises an acute neurodegenerative disease, a subacute neurodegenerative disease or a chronic neurodegenerative disease.

8. The method of claim 7, wherein the acute neurodegenerative disease comprises a stroke, a cerebral infarction, a cerebral hemorrhage, a head Injury or a spinal cord Injury;
the subacute neurodegenerative disease comprises a demyelinating disease, a paraneoplastic neurological syndrome, a subacute combined degeneration, a subacute necrotizing encephalitis, or a subacute sclerosing encephaliti; and
the chronic neurodegenerative disease comprises a senile dementia, a vascular dementia, a diffuse white matter disease (binswanger's disease), a dementia of endocrine or metabolic origin, a dementia due to head Injury or diffuse brain demage, an amnesia including dementia pugilistica and frontal lobe dementia, an Alzheimer's disease, a pick disease, a diffuse lewy body disease, a progressive supranuclear palsy (steele-richardson-olszewksi syndrome), a multiple system degeneration (shy-drager syndrome), a neurodegeneration related to Chronic epilepsy syndrome, an amyotrophic lateral sclerosis, a motor incoordination, a corticobasal degeneration, an Amyotrophic lateral sclerosis-parkinsonism/dementia complex of Guam, a subacute sclerosing encephalitis, a Huntington's disease, a Parkinson's disease, a synucleinopathy, a primary progressive aphasia, a striatonigral degeneration, a Machado-Joseph disease/spinocerebellar ataxia, a motor neuron cell disease including an olivopontocerebellar degeneration, a Gilles de la tourette disease, a bulbar and pseudobulbar paralysis, a spinal cord and spinobulbar amyotrophy (Kennedy Disease), a multiple sclerosis, a primary lateral sclerosis, a hereditary spastic paraplegia, a Werdnig-Hoffman disease, a Kugelberg-Welander disease, a tay-sachs disease, a sandhoff disease, a hereditary spastic disease, a Wohlfart-Kugelberg-Welander disease, a spastic paraplegia, a progressive multifocal leukoencephalopathy, a hereditary autonomic dysfunction (riley-day syndrome), a creutzfeldt-jakob disease, a gerstmann-strauissler-scheinker disease, a prion disease including a kuru disease and a fatal familial insomnia, or a cerebral palsy.

9. The method of claim 1, wherein the extract is prepared by a process comprising providing an adipose tissue stem cell; preparing a cell suspension of the adipose tissue stem cell by lysing, disrupting or permeabilizing the stem cell; and fractionating the cell suspension into a soluble and an insoluble fraction.

10. The method of claim 9, wherein the suspension is prepared by the lysis wherein the lysis is performed by a sonification and the stem cell is pretreated with a proteinase and washed with a buffer before the lysis.

11. A method for activating pCREB-PGC-1alpha pathway comprising administering an effective amount of adipose stem cell extract or the composition comprising the same to a subject in need thereof or cells.

12. A method for regulating an inflammatory response comprising administering an effective amount of adipose stem cell extract or the composition comprising the same to a subject in need thereof or cells.

13. A method for suppressing a neuronal cell-death comprising administering an effective amount of adipose stem cell extract or the composition comprising the same to a subject in need thereof or cells.

14. A pharmaceutical composition for treating or preventing a neurologic disease. comprising an adipose stem cell extract and a pharmaceutically acceptable carrier

15. The composition of claim 14, wherein the pharmaceutical composition activates pCREB-PGC-1alpha pathway, regulates inflammatory response or suppresses neuronal cell-death.

16. The composition of claim 14, wherein the stem cell is autologous adipose stem cell, heterologous adipose stem cell, or allogenic adipose stem cell.

17. The composition of claim 14, wherein the extract comprises soluble and/or insoluble fraction, wherein the soluble fraction comprises a protein, polypeptide or peptide.

18. The composition of claim 14, wherein the active ingredient in the extract is a protein, polypeptide or peptide.

19. The composition of claim 14, wherein the neurologic disease comprises an acute neurodegenerative disease, a subacute neurodegenerative disease or a chronic neurodegenerative disease.

20. The composition of claim 19, wherein the acute neurodegenerative disease comprises a stroke, a cerebral infarction, a cerebral hemorrhage, a head Injury or a spinal cord Injury;
the subacute neurodegenerative disease comprises a demyelinating disease, a paraneoplastic neurological syndrome, a subacute combined degeneration, a subacute necrotizing encephalitis, or a subacute sclerosing encephaliti; and
the chronic neurodegenerative disease comprises a senile dementia, a vascular dementia, a diffuse white matter disease (binswanger's disease), a dementia of endocrine or metabolic origin, a dementia due to head Injury or diffuse brain demage, an amnesia including dementia pugilistica and frontal lobe dementia, an Alzheimer's disease, a pick disease, a diffuse lewy body disease, a progressive supranuclear palsy (steele-richardson-olszewksi syndrome), a multiple system degeneration (shy-drager syndrome), a neurodegeneration related to Chronic epilepsy syndrome, an amyotrophic lateral sclerosis, a motor incoordination, a corticobasal degeneration, an Amyotrophic lateral sclerosis-parkinsonism/dementia complex of Guam, a subacute sclerosing encephalitis, a Huntington's disease, a Parkinson's disease, a synucleinopathy, a primary progressive aphasia, a striatonigral degeneration, a Machado-Joseph disease/spinocerebellar ataxia, a motor neuron cell disease including an olivopontocerebellar degeneration, a Gilles de la tourette disease, a bulbar and pseudobulbar paralysis, a spinal cord and spinobulbar amyotrophy (Kennedy Disease), a multiple sclerosis, a primary lateral sclerosis, a hereditary spastic paraplegia, a Werdnig-Hoffman disease, a Kugelberg-Welander disease, a tay-sachs disease, a sandhoff disease, a hereditary spastic disease, a Wohlfart-Kugelberg-Welander disease, a spastic paraplegia, a progressive multifocal leukoencephalopathy, a hereditary autonomic dysfunction (riley-day syndrome), a creutzfeldt-jakob disease, a gerstmann-strauissler-scheinker disease, a prion disease including a kuru disease and a fatal familial insomnia, or a cerebral palsy.


SEQUENCE LISTING (4 sequences)

SEQ ID NO: 1 - DNA, 25 bp, Artificial Sequence
  Sense primer for TNF alpha
tactgaacttcggggtgattggtcc

SEQ ID NO: 2 - DNA, 24 bp, Artificial Sequence
  Antisense primer for TNF alpha
cagccttgtcccttgaagagaacc

SEQ ID NO: 3 - DNA, 21 bp, Artificial Sequence
  Sense primer for IL-6
cttgggactgatgttgttgac

SEQ ID NO: 4 - DNA, 20 bp, Artificial Sequence
  Antisense primer for IL-6
tctgaatgactctggctttg